(19) United States
(12) Patent Application Publication
(10) Pub. No.: US 2023/0106535 A1
(43) Pub. Date: Apr. 6, 2023

(54) TECHNIQUES FOR DETERMINING CALCIMIMETIC DRUG ACTIVITY

(71) Applicant: Fresenius Medical Care Holdings, Inc., Waltham, MA (US)

(72) Inventors: Gudrun Schappacher-Tilp, Graz (AT); Peter Kotanko, Waltham, MA (US)

(21) Appl. No.: 17/491,900

(22) Filed: Oct. 1, 2021

(51) Int. Cl. G16C 20/30; G16H 50/50

(52) U.S. Cl. CPC G16C 20/30; G16H 50/50

(57) ABSTRACT

The described technology may include processes to model parathyroid gland (PTG) functionality and/or calcimimetic administration to healthy subjects and/or patients with a health abnormality that affects PTG function. In one embodiments, a computer-implemented method of calcimimetic analysis of PTG functionality may include accessing a calcimimetic model configured to simulate a functionality of a PTG of at least one patient, the calcimimetic model comprising at least one of a pharmacokinetic model or a pharmacodynamic model, providing a calcimimetic administration of a calcimimetic to the at least one patient via the calcimimetic model according to an administration process, and determining calcimimetic information based on the calcimimetic administration via the calcimimetic model for the at least one patient, the calcimimetic information configured to indicate an efficacy of the calcimimetic administration. Other embodiments are described.

### FIELD

The disclosure generally relates to processes for pharmacokinetic and/or pharmacodynamic modeling of calcimimetic compounds, including, without limitation, etelcalcetide, in the human body to determine healthcare information and/or administer treatment to patients.

### BACKGROUND

The concentration of extracellular ionized calcium is maintained within a narrow physiologic range via a biological system of negative and positive feedback regulators involving the major organs that transport calcium and phosphate, including the intestine, kidneys, bones, and the endocrine glands with the parathyroid gland (PTG) as the most prominent regulator. In patients with certain abnormal health conditions, such as chronic kidney disease (CKD), the loss of the regulatory kidney function triggers a cascade of processes eventually leading to disruptions of the ionized calcium regulatory system.

Clinical studies are an important tool for understanding the ionized calcium regulatory system in patients, particularly those with abnormal health conditions. However, clinical studies are expensive, time-consuming, and resource-intensive. Accordingly, virtual models of biological systems may be used in some situations to evaluate functionality and treatments without the need for real-world patients, regulations, and cost. Although some conventional models have been described for modeling PTG functionality, such modeling is limited and does not accurately model actual patients, particularly those with health conditions. For example, conventional techniques are limited because they do not provide processes for effectively simulating different administration schemes. In another example, conventional techniques do not accurately reflect PTG functionality in patient populations with health abnormalities that effect the PTG, such as CKD.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one embodiment, a computer-implemented method of calcimimetic activity analysis of the parathyroid gland (PTG) may include, via a processor of a computing device, accessing a calcimimetic model configured to simulate a functionality of a PTG of at least one patient, the calcimimetic model including at least one of a pharmacokinetic model or a pharmacodynamic model, providing a calcimimetic administration of a calcimimetic to the at least one patient via the calcimimetic model according to an administration process, and determining calcimimetic information based on the calcimimetic administration via the calcimimetic model for the at least one patient, the calcimimetic information configured to indicate an efficacy of the calcimimetic administration.

In some embodiments of the computer-implemented method, the calcimimetic may include etelcalcetide.

In various embodiments of the computer-implemented method, the calcimimetic administration may include at least one dose titration process for the calcimimetic. In exemplary embodiments of the computer-implemented method, the at least one dose titration process may include one or more of adjusting a dose of the calcimimetic on a constant time span, holding calcimimetic administration responsive to a calcium concentration being below a hold threshold, reducing a dose of the calcimimetic responsive to a calcium concentration being below a reduce threshold, or raising a calcimimetic dose responsive to a PTH concentration being within a threshold range.

In various embodiments of the computer-implemented method, the pharmacokinetic model may be configured to simulate pharmacokinetic functionality of the calcimimetic for the at least one patient, the calcimimetic information for the pharmacokinetic model may include a calcimimetic concentration. In some embodiments of the computer-implemented method, the calcimimetic may include etelcalcetide and the calcimimetic information for the pharmacokinetic model may include at least one of intact etelcalcetide, etelcalcetide biotransforms, or peripheral compartment etelcalcetide.

In various embodiments of the computer-implemented method, the calcimimetic information may be determined according to at least one of the following:

$$\frac{{dC}_{i}}{dt} = - ( \frac{{CL}_{u}}{{V}_{i}} + \frac{{C}_{e l}}{{V}_{i}} + {k}_{c} + {χ}_{HD} \cdot \frac{Q}{{V}_{i}} \cdot E ) \cdot {C}_{p} + {k}_{d c} \cdot {C}_{B i o}$$

$$\frac{{dC}_{Bio}}{dt} = - ( \frac{{CL}_{u}}{{V}_{bio}} \cdot Φ + \frac{{C}_{f}}{{V}_{bio}} + {k}_{p t} + {k}_{d c} + {χ}_{HD} \cdot \frac{Q}{{V}_{bio}} \cdot E \cdot Φ ) \cdot {C}_{Bio} + {k}_{c} \cdot {C}_{i} + {k}_{tp} \cdot {C}_{per}$$

$$\frac{{dC}_{per}}{dt} = - {k}_{tp} \cdot {C}_{per} + {k}_{p t} \cdot {C}_{Bio} ,$$

where C denotes a calcimimetic, i denotes intact calcimimetic, bio denotes calcimimetic biotransforms, and per denotes a peripheral compartment, where Q is the plasma flow during dialysis, where Φ denotes a fraction of filterable biotransforms, where E is a dialysis extraction ratio for the calcimimetic, where Vᵢ is a volume of distribution in a central compartments for intact calcimimetic, where V_{bio} is a volume of distribution in the central compartments for calcimimetic biotransforms, where χ_{HD}=1 if there is a dialysis session ongoing and χ_{HD}=0 if there is not a dialysis session ongoing, where k_{c} is a conjugation rate constant, where k_{dc} is a deconjugation rate constant, where kₚₜ is a transfer from plasma to peripheral compartment rate constant, where kₜₚ is a transfer from peripheral compartment to central compartment rate constant, where C_{f} is an elimination rate of intact calcimimetic in feces, where CLᵤ is a urinal clearance, and where Cₑₗ is an elimination rate of intact calcimimetic in plasma.

In some embodiments of the computer-implemented method, the pharmacodynamic model may be configured to simulate pharmacodynamic functionality of the calcimimetic for the at least one patient, the calcimimetic information for the pharmacokinetic model may include at least one of a parathyroid (PTH) concentration, a calcium concentration, or a phosphate concentration.

In exemplary embodiments of the computer-implemented method, the method may include determining at least one treatment recommendation based on the calcimimetic information. In some embodiments of the computer-implemented method, the method may include determining at least one clinical trial based on the calcimimetic information.

In one embodiment, an apparatus may include at least one processor and a memory coupled to the at least one processor, the memory comprising instructions that, when executed by the at least one processor, cause the at least one processor to access a calcimimetic model configured to simulate a functionality of a PTG of at least one patient, the calcimimetic model including at least one of a pharmacokinetic model or a pharmacodynamic model, provide a calcimimetic administration of a calcimimetic to the at least one patient via the calcimimetic model according to an administration process, and determine calcimimetic information based on the calcimimetic administration via the calcimimetic model for the at least one patient, the calcimimetic information configured to indicate an efficacy of the calcimimetic administration.

In some embodiments of the apparatus, the calcimimetic may include etelcalcetide.

In various embodiments of the apparatus, the calcimimetic administration may include at least one dose titration process for the calcimimetic. In exemplary embodiments of the apparatus, the at least one dose titration process may include one or more of adjusting a dose of the calcimimetic on a constant time span, holding calcimimetic administration responsive to a calcium concentration being below a hold threshold, reducing a dose of the calcimimetic responsive to a calcium concentration being below a reduce threshold, or raising a calcimimetic dose responsive to a PTH concentration being within a threshold range.

In some embodiments of the apparatus, the pharmacokinetic model may be configured to simulate pharmacokinetic functionality of the calcimimetic for the at least one patient, the calcimimetic information for the pharmacokinetic model comprising a calcimimetic concentration. In various embodiments of the apparatus, the calcimimetic may include etelcalcetide and the calcimimetic information for the pharmacokinetic model comprising at least one of intact etelcalcetide, etelcalcetide biotransforms, or peripheral compartment etelcalcetide.

In some embodiments of the apparatus, the calcimimetic information may be determined according to at least one of the following:

$$\frac{{dC}_{i}}{dt} = - ( \frac{{CL}_{u}}{{V}_{i}} + \frac{{C}_{e l}}{{V}_{i}} + {k}_{c} + {χ}_{HD} \cdot \frac{Q}{{V}_{i}} \cdot E ) \cdot {C}_{p} + {k}_{d c} \cdot {C}_{B i o}$$

$$\frac{{dC}_{Bio}}{dt} = - ( \frac{{CL}_{u}}{{V}_{bio}} \cdot Φ + \frac{{C}_{f}}{{V}_{bio}} + {k}_{p t} + {k}_{d c} + {χ}_{HD} \cdot \frac{Q}{{V}_{bio}} \cdot E \cdot Φ ) \cdot {C}_{Bio} + {k}_{c} \cdot {C}_{i} + {k}_{tp} \cdot {C}_{per}$$

$$\frac{{dC}_{per}}{dt} = - {k}_{tp} \cdot {C}_{per} + {k}_{p t} \cdot {C}_{Bio} ,$$

where C denotes a calcimimetic, i denotes intact calcimimetic, bio denotes calcimimetic biotransforms, and per denotes a peripheral compartment, where Q is the plasma flow during dialysis, where Φ denotes a fraction of filterable biotransforms, where E is a dialysis extraction ratio for the calcimimetic, where Vᵢ is a volume of distribution in a central compartments for intact calcimimetic, where V_{bio} is a volume of distribution in the central compartments for calcimimetic biotransforms, where χ_{HD}=1 if there is a dialysis session ongoing and χ_{HD}=0 if there is not a dialysis session ongoing, where k_{c} is a conjugation rate constant, where k_{dc} is a deconjugation rate constant, where kₚₜ is a transfer from plasma to peripheral compartment rate constant, where kₜₚ is a transfer from peripheral compartment to central compartment rate constant, where C_{f} is an elimination rate of intact calcimimetic in feces, where CLᵤ is a urinal clearance, and where Cₑₗ is an elimination rate of intact calcimimetic in plasma.

In various embodiments of the apparatus, the pharmacodynamic model may be configured to simulate pharmacodynamic functionality of the calcimimetic for the at least one patient, the calcimimetic information for the pharmacokinetic model comprising at least one of a parathyroid (PTH) concentration, a calcium concentration, or a phosphate concentration.

In exemplary embodiments of the apparatus, the instructions, when executed by the at least one processor, to cause the at least one processor to determine at least one treatment recommendation based on the calcimimetic information. In some embodiments of the apparatus, the instructions, when executed by the at least one processor, to cause the at least one processor to determine at least one clinical trial based on the calcimimetic information.

### BRIEF DESCRIPTION OF THE DRAWINGS

By way of example, specific embodiments will now be described, with reference to the accompanying drawings, in which:

FIG. 1 illustrates a first exemplary operating environment in accordance with the present disclosure;

FIG. 2 depicts pharmacokinetics information for a pharmacokinetic model according to some embodiments;

FIG. 3 depicts pharmacokinetics information for a pharmacokinetic model according to some embodiments;

FIG. 4 depicts pharmacokinetics information for a pharmacokinetic model according to some embodiments;

FIG. 5 depicts pharmacokinetics information for a pharmacokinetic model according to some embodiments;

FIG. 6 depicts pharmacokinetics information for a pharmacokinetic model according to some embodiments;

FIG. 7 depicts pharmacokinetics information for a pharmacokinetic model according to some embodiments;

FIG. 8 depicts pharmacokinetics information for a pharmacokinetic model according to some embodiments;

FIG. 9 depicts model information for simulating etelcalcetide administration in healthy patients according to some embodiments;

FIG. 10 depicts model information for simulating etelcalcetide administration in HD patients according to some embodiments;

FIGS. 11A-11C depict illustrative hemodialysis information for in-center hemodialysis patients;

FIG. 12 depicts an illustrative example of a discretization blood flow distribution according to some embodiments;

FIG. 13 depicts calcimimetic model information according to some embodiments;

FIGS. 14-16 depict calcimimetic information generated via calcimimetic models according to some embodiments;

FIG. 17 depicts simulation results for an illustrative calcimimetic model according to some embodiments;

FIG. 18 depicts simulation results for an illustrative calcimimetic model according to some embodiments;

FIG. 19 depicts simulation results for an illustrative calcimimetic model according to some embodiments;

FIG. 20 depicts simulation results for an illustrative calcimimetic model according to some embodiments;

FIG. 21 depicts simulation results for an illustrative calcimimetic model according to some embodiments;

FIG. 22 depicts simulation results for an illustrative calcimimetic model according to some embodiments

FIG. 23 depicts experimental results used to calibrate a phosphate-adjusted calcimimetic model;

FIG. 24 depicts calcimimetic information generated via a phosphate-adjusted calcimimetic model according to some embodiments

FIG. 25 depicts calcimimetic model information according to some embodiments;

FIG. 26 depicts calcimimetic information generated via calcimimetic models according to some embodiments;

FIG. 27 depicts calcimimetic information generated via calcimimetic models according to some embodiments;

FIG. 28 illustrates an example of an operating environment 2800 that may be representative of some embodiments;

FIG. 29 depicts the functionality of illustrative calcimimetic models according to some embodiments

FIGS. 30A and 30B depict illustrative calcimimetic models according to some embodiments; and

FIG. 31 illustrates an embodiment of an exemplary computing architecture.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and convey the scope of the subject matter to those skilled in the art. In the drawings, like numbers refer to like elements throughout.

Parathyroid hormone (PTH) secretion is primarily regulated by the ionized calcium concentration (Ca or Ca²⁺) as well as phosphate concentration in the extracellular fluid and calcitriol, the bioactive form of vitamin D (D or 1.25D). Metabolic disturbances in patients with chronic kidney disease (CKD) and/or end-stage kidney disease (ESKD) may lead to alterations in parathyroid gland (PTG) biology and/or function. A hallmark of CKD is secondary hyperparathyroidism, characterized by an increased production and release of PTH, reduced expression of calcium-sensing on the surface of parathyroid cells as well as vitamin D receptors in parathyroid cells, and hyperplasia and hypertrophy of these cells. These alterations happen on different time-scales and influence each other, thereby triggering a highly-complex cascade of negative and positive feedback loops. Due to this complexity, models may be used, inter alia, to break down the patterns of the multi-dimensional cascade of processes and enable the detailed study of subsystems.

In some embodiments, a calcimimetic analysis process may use one or more calcimimetic models to analyze the administration of one or more calcimimetic drugs targeting the PTG. In exemplary embodiments, the calcimimetic may be or may include etelcalcetide (Ete) and/or cinacalcet. In one embodiment, the calcimimetic may include etelcalcetide. Although etelcalcetide and cinacalcet are used in examples in the present disclosure, embodiments are not so limited, as other calcimimetic and/or non-calcimimetic compounds may be used according to some embodiments.

In various embodiments, the calcimimetic analysis process may use one or more calcimimetic models that may include a pharmacokinetic model and/or a pharmacodynamic model. In some embodiments the calcimimetic models may be used in combination with a PTG model. In some embodiments, the pharmacokinetic model may operate to mimic, model, simulate, emulate, determine, evaluate, predict, or otherwise process a calcimimetic and/or forms thereof (for instance, biotransforms) for a calcimimetic administration to a virtual patient and/or population of patients. In some embodiments, patients may include dialysis patients, such as hemodialysis (HD) patients. In various embodiments, the pharmacokinetic model may include HD elements, such as blood flow, hematocrit, compound filtering, and/or the like. In some embodiments, the pharmacokinetic model may be a multi-compartmental model, for example, including a central compartment (for instance, a compartment of calcimimetic administration), one or more peripheral compartments, and/or the like.

In some embodiments, the calcimimetic models may generate calcimimetic information, which may include, for example, pharmacokinetic information and/or pharmacodynamic information. In exemplary embodiments, the pharmacokinetic model may determine pharmacokinetic information associated with administration of a calcimimetic. In some embodiments, the pharmacokinetic information may include concentrations or other information associated with various types or forms of one or more calcimimetic compounds including, without limitation, intact calcimimetic and/or biotransform calcimimetic, and/or a location of the calcimimetic compound, such as a concentration within a particular compartment.

In some embodiments, the pharmacodynamic model may operate to mimic, model, simulate, emulate, determine, evaluate, predict, or otherwise generate pharmacodynamic information associated with administration of a calcimimetic to a patient and/or population of patients. In various embodiments, the pharmacodynamic information may include the concentration of or other information associated with target compounds. In some embodiments, the target compounds may include PTH, calcium (Ca), and/or phosphate (P). In various embodiments, the pharmacodynamic model may operate to predict PTH reduction, PTH levels, phosphate information (for instance, mean phosphate concentration), Ca information (for instance, ionized Ca reduction). In exemplary embodiments, the pharmacodynamic model may be used for HD patients and/or non-HD (for instance, “normal” or “healthy”) subjects.

In various embodiments, the calcimimetic analysis process may use the pharmacodynamic model and the PTG model in combination to determine the pharmacodynamic information. In some embodiments, the PTG model may be the same or similar to the PTG models described in U.S. patent application Ser. No. 16/655,873, filed on Oct. 17, 2019, titled “Techniques for Modeling Parathyroid Gland Functionality and Calcimimetic Drug Activity,” and published Apr. 23, 2020 as U.S. Patent Application Publication No. 2020/0126632, the contents of which are hereby incorporated by reference as if fully set forth herein. In some embodiments, the pharmacodynamic model may operate to model etalcalcetide and the PTG model may operate to model cinacalcet.

The concentration of extracellular ionized calcium is maintained within a narrow physiologic range by a biological system of negative and positive feedback regulators involving the major organs that transport calcium and phosphate, for instance, the intestine, kidneys, bones, endocrine glands such as most prominently, the PTG. Under normal conditions, extracellular ionized calcium concentration (Ca²⁺) is maintained within a narrow range of only about 1-2%.

A key endocrine regulator for serum Ca²⁺ is PTH, which increases serum calcium levels by enhancing renal tubular calcium reabsorption, stimulating net bone resorption, and increasing the production of activated vitamin D (1,25-dihydroxyvitamin D3 or 1.25(OH)2D3) which increases net intestinal calcium absorption. PTH is produced, stored, and eventually released by the PTG. The PTG consists of two cell populations: active secretory cells and quiescent cells, which are able to proliferate or undergo apoptosis. Secretory active cells produce, store, and release PTH. PTG activity itself is mainly regulated by the calcium-sensing receptors (CaSR) on the surface of PTG cells.

The CaSR is integrally involved in PTG function and biology and, therefore, the key to understanding pathologies like secondary hyperparathyroidism. The activation of the CaSR by the binding of Ca²⁺ initiates several signaling pathways which down-regulate PTH release, production, and PTG cellular proliferation rates. Moreover, the CaSR regulates PTH mRNA stability, thereby mediating its rate of degradation. Besides controlling PTH secretion, synthesis, degradation and PTG proliferation, the CaSR upregulates vitamin D receptor (VDR) expression, and vice versa.

The PTG adapts swiftly to conditions requiring enhanced PTH secretion, such as hypocalcemia. The different adaptive responses manifest on significantly different time scales, reaction times ranging from minutes to hours, to days, and weeks. For instance, in the case of an acute hypocalcemia, the PTG quickly responds by releasing stored PTH within seconds to minutes. If hypocalcemia persists, intracellular PTH degradation rate declines within 20 minutes, thereby increasing the amount of intact PTH that can be released. If normocalcemia is still not attained, the PTH production rate increases within an hour. Subsequently, the PTG will augment its cellular proliferation rate within two days. In chronic hypocalcemia the enhanced proliferation rate will lead to hyperplasia whereby the PTG mass increases 10-100-fold or more.

In patients with chronic kidney disease (CKD) the loss of the regulatory kidney function triggers a cascade of processes eventually leading to secondary hyperparathyroidism, an abnormality characterized by increased PTH synthesis and secretion, and PTG cell proliferation. One hallmark of CKD is the impaired renal synthesis of the principal bioactive form of vitamin D, 1,25-dihydroxyvitamin D3 (1.25D), and the significantly impaired renal clearance of phosphate. Both 1.25D and phosphate significantly influence CaSR regulation, and thereby PTH synthesis and release, and PTG proliferation. There is a positive feedback loop between the CaSR and vitamin D receptor (VDR) expression, and a suppression of CaSR expression at high phosphate levels. The pathological effects exerted by high phosphate levels are significant. For example, studies in uremic rats have shown that a high phosphate diet enhances PTG proliferation within a few days. Low calcium intake has similar results but the required time is significantly longer. Importantly, the positive feedback loop of CaSR and VDR is effectively diminished in the presence of high phosphate levels.

In CKD, PTH stimulates bone resorption over bone formation, resulting in a net release of Ca²⁺ and phosphate from the bone into the systemic circulation. As a consequence, PTH and phosphate levels increase even further and expression of CaSR and VDR is depressed, thus lowering the sensitivity of the PTG to Ca²⁺ and 1.25D. The altered biology eventually results in PTG hyperplasia and secondary hyperparathyroidism.

In general, etelcalcetide is a calcimimetic drug that may be administered for the treatment of bone disorders in dialysis patients. For example, etelcalcetide may be administered intravenously to HD patients during a dialysis treatment. Etelcalcetide functions by binding to and activating the CaSR in the PTG. With respect to in vitro pharmacokinetics, etelcalcetide forms various products in the human body. The major biotransformation product is serum albumin peptide conjugate (SAPC) with a high molecular weight. The remaining biotransforms are products with lower molecular weight (LwEte). The formations are reversible and, as such, a dynamic equilibrium may be assumed. Etelcalcetide is mainly biotransformed by disulfide exchange of the L-cysteine with thiols in the blood, primarily with serum albumin.

Experimental in vitro pharmacokinetic studies have determined kinetics estimates for biotransformation of etelcalcetide and HD dynamics within patients. Non-limiting examples of such studies are presented in Edson et al., “Determination of Etelcalcetide Biotransformation and Hemodialysis Kinetics to Guide the Timing of Its Dosing,” KIDNEY INTERNATIONAL REPORTS 1:24-33 (2016), the contents of which are incorporated by reference as if fully set forth herein.

In healthy subjects, etelcalcetide is mainly excreted in urine. During hemodialysis (HD), small molecules are excreted including low-molecular weight biotransformation products of etelcalcetide (for instance, <2 kilodaltons (kDa)), while SAPC is retained because of its high molecular weight. Studies have indicated, for example, that HD cleared approximately 70% of the etelcalcetide dose within 15 minutes, almost all of cleared as intact etelcalcetide. The rate of conversion from SAPC to etelcalcetide is roughly 16-fold faster than the conversion of etelcalcetide to SAPC. The rate of conversion from SAPC to etelcalcetide is approximately 200 fold slower than the dialytic rate constant. Therefore, an administration before HD would clear etelcalcetide quickly. If etelcalcetide is in equilibrium with SAPC, the dialysis of etelcalcetide-related components is slow. The HD dynamics corresponds to a HD clearance rate of 36 ml/min or approximately 70% of blood flow rate.

Experimental clinical pharmacokinetic studies have determined kinetics estimates of etelcalcetide within patients. A non-limiting example of a clinical pharmacokinetic study in healthy subjects includes Shen et al., “A Pharmacokinetic/Pharmacodynamic Model for AMG 416, a Novel Calcimimetic Peptide, Following a Single Intravenous Dose in Healthy Subjects,” JOURNAL OF CLINICAL PHARMACOLOGY 54:1125-1133 (2014) (“Shen Study”), the contents of which are incorporated by reference as if fully set forth herein. Studies have indicated that etelcalcetide plasma concentration declines in a tri-exponential manner and has a terminal half-life of around 20 hours in healthy subjects.

Etelcalcetide has a significantly prolonged terminal half-life in patients with CKD treated by HD. Studies have estimated the half-life to be in the range of 50 days in patients on intermittent HD. However, methods of estimation and thereby, estimates, vary greatly between the different studies. For example, other studies have indicated intradialytic elimination half-life estimates between 82 and 175 hours. In addition, studies have reported that approximately 90% of etelcalcetide undergoes biotransformation, roughly 60% biotransformation resulting in the (reversible) formation of SAPC. The later products are too large for passing the dialyzer membrane. SAPC therefore may act as a reservoir for etelcalcetide after hemodialysis. While etelcalcetide concentration (for instance, plasma concentration) has been shown to drop during a HD session, it quickly recovers; pre-dialysis concentrations decline over time. A non-limiting example of a study demonstrating the clinical pharmacokinetics and pharmacodynamics of etelcalcetide includes Wu et al., “Clinical Pharmacokinetics and Pharmacodynamics of Etelcalcetide, a Novel Calcimimetic for Treatment of Secondary Hyperparathyroidism in Patients With Chronic Kidney Disease on Hemodialysis,” Journal of Clinical Pharmacology, 58:717-726, 2018 (“Wu Journal of Clinical Pharmacology”), the contents of which are hereby incorporated by reference as if fully set forth herein.

Models have been developed to simulate PTH biological function. For example, conventional PTH modeling techniques have used three-compartmental models using various parameters to estimate an amount of intact etelcalcetide in a central, shallow, and deep peripheral compartments. However, parameter estimates vary greatly between HD patients and healthy subjects. Due to the almost constant concentration in HD patients after the first hours post-administration, fitting certain parameters such as terminal half-life and area-under-the-concentration-time-curve (AUC) leads to inaccurate estimates. Other models (for instance, Wu Journal of Clinical Pharmacology, referenced above) have been configured based on the premise that etelcalcetide is rapidly bio-transformed (BTx) following intravenous administration and relies on certain assumptions regarding etelcalcetide elimination paths. However, the reliance on unknown etelcalcetide elimination paths leads to non-reliable results for such modeling techniques. In addition, such models have shown to be a poor fit for etelcalcetide pharmacokinetics in healthy patients. Additional models have relied on a drug disposition model, for instance, as described in Wu et al., “Drug Disposition Model of Radiolabeled Etelcalcetide in Patients with Chronic Kidney Disease and Secondary Hyperparathyroidism on Hemodialysis,” Journal of Pharmacokinetics and Pharmacodynamics, 44:43-53, 2017, the contents of which are hereby incorporated by reference as if fully set forth herein. However, such drug disposition models are a poor fit for multiple predictions, including, without limitation, for days after a etelcalcetide administration (for example, a single administration of 10 milligrams (mg) of etelcalcetide), short-term predictions, and multiple administrations. In addition, drug disposition models have generated estimated data that does not agree with corresponding clinical data.

Accordingly, conventional systems for modeling PTG biological activity, including calcimimetic activity, do not provide sufficient estimation accuracy to be useful for modeling actual clinical studies. Therefore, some embodiments may provide calcimimetic models that are accurate, efficient, and provide relevant data for modeling clinical studies. For example, as described in the present disclosure, calcimimetic models according to various embodiments are able to generate predictive data with a high correspondence to actual clinical data, demonstrating their predictive capabilities to provide useful modeling techniques.

Due to the complexity of the adaptation of PTG biology in patients with CKD on hemodialysis, some embodiments may provide a calcimimetic analysis process, which may include one or more calcimimetic functionality models to analyze, evaluate, simulate, or otherwise examine the PTG under normal and/or abnormal health conditions to determine optimized treatment strategies. The calcimimetic analysis process according to some embodiments may provide a key element of an in-silico model of calcimimetic treatment regimens in patients, including patients with PTG abnormalities, such as CKD and HD patients. Accordingly, calcimimetic analysis processes according to some embodiments may operate to generate predictions about the development of PTG abnormalities and/or treatment of PTG abnormalities with calcimimetic compounds.

While there are various models of PTG activity in humans, conventional techniques fail to capture key adaptation mechanisms of the complex network regulating PTG functionality, PTH activity, and/or calcimimetic activity, particularly in patients with CKD or other health abnormalities that may affect PTG functionality. In addition, due to the vast clinical use of calcimimetics, such as etelcalcetide and/or cinacalcet, a realistic model of PTG activity should feature the use of calcimimetics and their effects. Furthermore, to be of clinical use the calcimimetic model should be readily adaptable to various conditions, such as hepatic impairment enhancing etelcalcetide and/or cinacalcet exposure, and should be able to reflect different administration scenarios, such as different dosage and/or titration techniques.

Accordingly, some embodiments may provide a calcimimetic analysis process that may include various models to simulate the functionality of the PTG and/or calcimimetic activity based on, for example, CaSR expression and activity regulated by ionized calcium (Ca or Ca²⁺), phosphate (P), and vitamin D (D or 1.25D). Some embodiments may provide multi-compartment calcimimetic models based on physiological considerations capturing all major pharmacokinetics parameters and/or pharmacodynamics parameters of a calcimimetic compound, such as etelcalcetide.

Therefore, calcimimetic analysis processes according to some embodiments may provide multiple technological advantages and technical features over conventional systems, including improvements to computing technology. One non-limiting example of a technological advantage may include modeling the various mechanisms ensuring enhanced PTH levels acting on different time scales, thereby allowing predictions for both rapid responses, for example, in the case of induced acute hypocalcemia, and long-term adaptations reflecting the transition of the healthy PTG into a hyperplastic gland with reduced sensitivity to Ca²⁺ and 1.25D. Another non-limiting example of a technological advantage may include providing a calcimimetic model operative to provide intuitive individualization to various conditions or administration regiments and omit numerical instabilities in order to be easily implemented in other physiological models (for instance, a PTG functionality model according to some embodiments). A further non-limiting example of a technological advantage may include providing a calcimimetic analysis process operative to generate treatment recommendations and/or determine research outcomes using virtual simulations of PTG functionality and/or calcimimetic administration without requiring clinical studies with actual patient participants.

Calcimimetic analysis processes according to some embodiments may provide improvements to computing technology and/or the technical field of CKD clinical research and/or medical treatment by providing realistic and accurate models of calcimimetic activity in patients that are not available using conventional technology. The field of CKD clinical research is improved by allowing researchers to have a powerful and accurate set of computer-based tools to determine in-silico calcimimetic treatment regimens for patients that have effective treatment outcomes. Such calcimimetic treatment regimens may then narrow the focus of the more expensive and time-consuming real-world clinical studies to those that have been demonstrated to be effective via the calcimimetic analysis processes, allowing researchers to avoid costly studies for treatments that are not sufficiently effective. In another example, the calcimimetic analysis processes may demonstrate treatment schemes, dosages, titration methods, and/or the like that may be applied for actual patient treatment. Accordingly, a calcimimetic treatment may be administered to a patient based on the results of a calcimimetic analysis processes according to some embodiments. Therefore, calcimimetic analysis processes according to various embodiments may be applied to the practical applications of clinical research and/or patient medical treatment. Other technological advantages are provided in this Detailed Description. Embodiments are not limited in this context.

In some embodiments, a calcimimetic model may be or may include a pharmacokinetic model. In various embodiments, a pharmacokinetic model may include certain assumptions, configurations, parameters, settings, and/or the like. For example, etelcalcetide can be biotransformed and the major part of the biotransforms may be SAPC which is not cleared by the kidneys or HD. Etelcalcetide and its biotransforms may exist in equilibrium, for example, after certain time duration to establish biotransform equilibrium. In another example, the pharmacokinetic model may include a central and one peripheral compartment for etelcalcetide. Since the albumin bound species is more abundant than the free etelcalcetide, the free fraction may be bound to albumin before it can be distributed to the peripheral compartment. Therefore, the pharmacokinetic model may assume that the biotransformed fraction is distributed, at least in part, to the peripheral compartment. In a further example, the pharmacokinetic model may assume that the biotransform of etelcalcetide is the main species excreted in feces. In an additional example, the pharmacokinetic model may be configured and may include model parameters such that a difference between healthy subjects and HD patients is the urinal clearance rate.

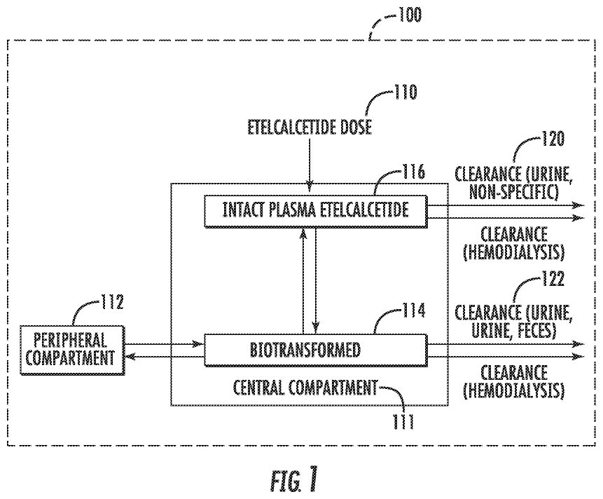

FIG. 1 depicts an illustrative pharmacokinetic model in accordance with the present disclosure. In some embodiments of the pharmacokinetic model 100, an etelcalcetide does 110 may be administered to a central compartment 111 where it may be biotransformed 114. Biotransforms 114 may be disposed of in a peripheral compartment 112 and/or cleared 122 through the kidneys, feces, and/or HD. Intact plasma etelcalcetide 116 may be cleared 120 by the kidneys or by HD and/or by a non-specific mechanism.

In some embodiments, the pharmacokinetic model may include the following:

$$\frac{d {C}_{i}}{d t} = - ( \frac{C {L}_{u}}{{V}_{i}} + \frac{{C}_{e l}}{{V}_{i}} + {k}_{c} + {χ}_{H D} \cdot \frac{Q}{{V}_{i}} \cdot E ) \cdot {C}_{i} + {k}_{d c} \cdot {C}_{B i o} \\ \frac{d {C}_{B i o}}{d t} = - ( \frac{C {L}_{u}}{{V}_{b i o}} \cdot Φ + \frac{{C}_{f}}{{V}_{b i o}} + {k}_{p t} + {k}_{d c} + {χ}_{H D} \cdot \frac{Q}{{V}_{b i o}} \cdot E \cdot Φ ) \cdot {C}_{B i o} + {k}_{c} \cdot {C}_{i} + {k}_{t p} \cdot {C}_{per} , and / or \\ \frac{{dC}_{p e r}}{d t} = - {k}_{t p} \cdot {C}_{per} + {k}_{p t} \cdot {C}_{B i o} ,$$

where i denotes intact etelcalcetide (which is decisive for pharmacodynamics), bio denotes biotransforms, and per denotes the peripheral compartment. Etelcalcetide may be administered to the intact etelcalcetide compartment. χ_{HD}=1 if there is a HD session ongoing and zero otherwise. Q is the plasma flow during dialysis (such as HD) (which is estimated by Q=Q_{B}·(1−HCT), where Q_{B} is the blood flow during the dialysis session and HCT is the hematocrit), where k_{c} is a conjugation rate constant, where k_{dc} is a deconjugation rate constant, where kₚₜ is a transfer from plasma to peripheral compartment rate constant, where kₜₚ is a transfer from peripheral compartment to central compartment rate constant, where C_{f} is an elimination rate of intact calcimimetic in feces, where CLᵤ is a urinal clearance, and where Cₑₗ is an elimination rate of intact calcimimetic in plasma. The constant Φ denotes the fraction of filterable biotransforms (≈8%) and E is the HD extraction ratio for etelcalcetide (≈0.385). Vᵢ and V_{bio} are the volume of distribution in the central compartments for intact etelcalcetide and the biotransforms, respectively.

Pharmacokinetics parameters and conjugation rates have been reported in various studies, such as the Shen Study, which indicate that there is a significant difference in reported pharmacokinetics in HD patients and in healthy patients. For example, the two non-terminal half-life values are greater in healthy patients than in HD patients. Accordingly, in some embodiments configured to simulate HD patients, data from HD patients may be used as the main or only reference (e.g., recovery in the dialysate and concentration decline within the first 2 hours).

In one experiment involving methods and systems according to some embodiments, the mean and SD values for AUC and terminal half-life presented in the Shen Study were used in order to validate the prediction ability of the pharmacokinetics model. Specifically, 400 reference AUC values were generated for the four dosage concentrations of 0.5 mg, 2 mg, 5 mg, and 10 mg in the Shen Study and 100 reference values t_{1/2} based on the mean values and standard errors in the Shen Study, assuming a normal distribution for these parameters. In some embodiments, calcimimetic analysis processes may use various optimization processes. For example, a pharmacokinetic model may include an optimization process that uses the following constraint optimizations, for example, to estimate model parameters for each set i of reference values:

min∥AUCⁱ−AUC_{Ref}ⁱ,t_{1/2}ⁱ−t_{1/2,Ref}ⁱ,VDⁱ−VD_{Ref}ⁱ∥.

AUC and t_{1/2} may be based on non-compartmental analysis (NCA). Simulated etelcalcetide plasma concentrations at various time points (for instance, 0.08, 0.17, 0.25, 0.33, 0.5, 1, 1.5, 2, 2.5, 3, 4, 6, 12, 24, 36, and 48 hours after dosing) may be used to calculate AUC and t_{1/2} based on NCA.

In some embodiments, AUCₙ (integral over plasma concentration) may be determined according to the following:

$${\int }_{0}^{n} C ( t ) dt ; and \\ \\ \\ \\ \\ \begin{matrix}AUC = {\int }_{0}^{∞} C ( t ) d t = {\int }_{0}^{∞} {\sum }_{i = 1}^{n} {c}_{i} {w}_{i}^{1} \cdot {e}^{{λ}_{i} \cdot t} d t \\ = {\sum }_{i = 1}^{n} {c}_{i} {w}_{i}^{1} {\int }_{0}^{∞} {e}^{{λ}_{i} \cdot t} d t \\ = - {\sum }_{i = 1}^{n} {c}_{i} {w}_{i}^{1} \frac{1}{{λ}_{i}}\end{matrix}$$

In some embodiments, each set of reference values may be treated as pharmacokinetics parameters from an individual subject, for example, to predict simultaneously the AUC for all etelcalcetide doses and the terminal half-life of one reference set with one set of model parameters. A model parameter set, such as the 100 model parameter sets based on the generated reference parameters, may be used to calculate the volume of distribution (VD) and the clearance (CL) for each set based on NCA.

In some embodiments, CL may be determined according to the following:

$$C L = \frac{D}{A U {C}_{∞}} .$$

In various embodiments, VD may be determined according to the following:

$${V}_{D} = D \frac{{\int }_{0}^{∞} t \cdot C ( t ) dt}{{( {\int }_{0}^{∞} C ( t ) d t )}^{2}} ; and \\ \\ \\ \\ \begin{matrix}{V}_{D} = D \frac{{\int }_{0}^{∞} t \cdot C ( t ) dt}{{( {\int }_{0}^{∞} C ( t ) d t )}^{2}} \\ = \frac{D}{A U {C}^{2}} \cdot {\int }_{0}^{∞} {\sum }_{i = 1}^{n} {c}_{i} {w}_{i}^{1} \cdot t \cdot {e}^{{λ}_{i} \cdot t} d t \\ = \frac{D}{A U {C}^{2}} \cdot {\sum }_{i = 1}^{n} {c}_{i} {w}_{i}^{1} {\int }_{0}^{∞} t \cdot {e}^{{λ}_{i} \cdot t} d t \\ = \frac{D}{A U {C}^{2}} \cdot {\sum }_{i = 1}^{n} ( \frac{1}{{λ}_{i}} t \cdot {e}^{{λ}_{i} \cdot t} ) {\vert}_{0}^{∞} - {\sum }_{i = 1}^{n} {c}_{i} {w}_{i}^{1} \frac{1}{{λ}_{i}} {\int }_{0}^{∞} t \cdot {e}^{{λ}_{i} \cdot t} d t \\ = \frac{D}{A U {C}^{2}} \cdot {\sum }_{i = 1}^{n} {c}_{i} {w}_{i}^{1} \frac{1}{{λ}_{i}^{2}}\end{matrix}$$

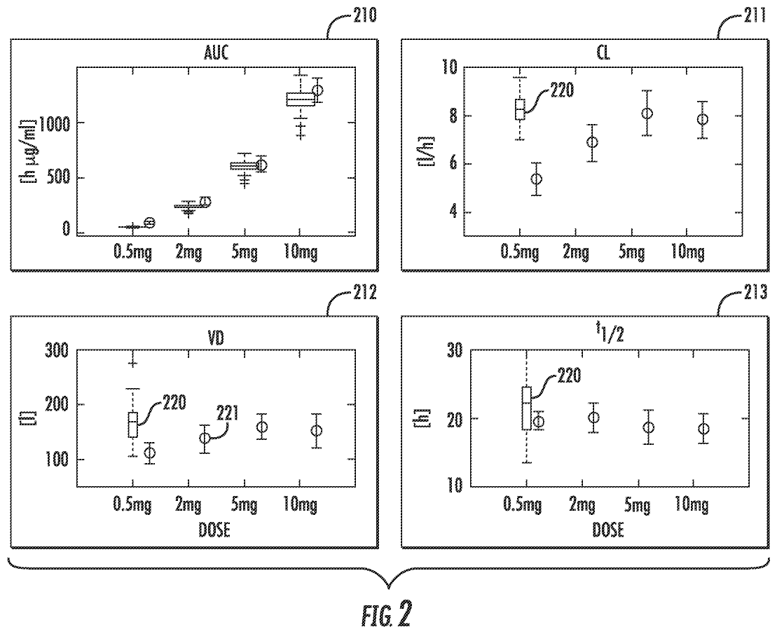

FIG. 2 depicts pharmacokinetics information for a pharmacokinetic model according to some embodiments. More specifically, FIG. 2 depicts results or pharmacokinetic information 220 (rectangle elements) generated via a pharmacokinetic model using an optimization process according to some embodiments determined based on a generated reference set (N=100). As shown in FIG. 2, a pharmacokinetic model may generate pharmacokinetic information 220 for various parameters, such as AUC 210, VD 211, CL 212, and/or t_{1/2} 213, based on the pharmacokinetic model and optimization process according to some embodiments. Clinical data 221 (circular elements) for published studies is depicted as a mean+/−standard deviation (SD). With respect to the clinical data 221, in theory, CL, VD, and t_{1/2} should be dose independent. However, clinical values 221 are based on different subjects for each dose; therefore, the values vary with the dose. Although there may be multiple pharmacokinetic information 220 elements or clinical values 221 in each graph 210-213, only one is labeled to simplify the figure.

The following Table 1 depicts parameters for the pharmacokinetic model based on the generated reference set (N=100):

TABLE 1

| Parameter | Geometric mean | | Geometric CV |
|---|---|---|---|
| CLᵤ | 4.6 | l/h | 5.9% |
| CLₙ | 3.4 · 10⁻¹ | l/h | 0.018% |
| CL_{f} | 0.12 | l/h | 0.022% |
| kₚₜ | 0.17 | 1/h | 16.6% |
| kₜₚ | 0.11 | 1/h | 22.3% |
| k_{c} | 3 | 1/h | 7.4% |

| | | |
|---|---|---|
| k_{dc} | 0.991/h | 18.23% |

As indicated in Table 1, the deconjugation and conjugation rate constants may show the highest variations, and renal clearance may be predominant in the pharmacokinetic model. In general, the coefficient of variation (CV) provides a measure or indication of the dispersion of data points in a data series around the mean. For example, CV may measure or indicate the extent of variability of data in a sample in relation to the mean of the population.

Calcimimetic models according to some embodiments, including pharmacokinetic models, may be used to determine pharmacokinetic information for various calcimimetic administration methods, including different doses. For example, pharmacokinetic models may be used to simulate long term pharmacokinetics of a single etelcalcetide administration (for instance, 10 mg) as well as the short term pharmacokinetics of single etelcalcetide administrations of different doses (for instance, 5 mg, 10 mg, 20 mg, 40 mg, and/or 60 mg).

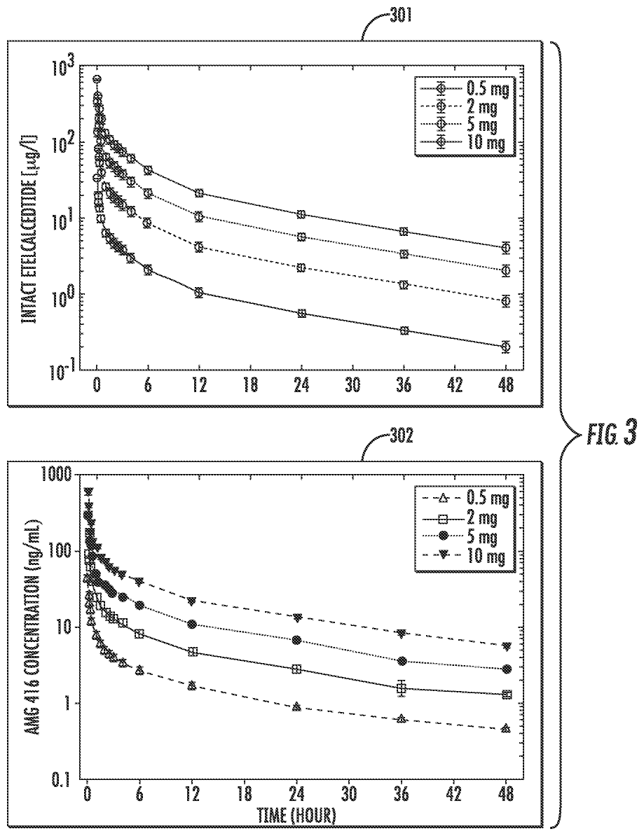

FIG. 3 depicts pharmacokinetics information for a pharmacokinetic model according to some embodiments. More specifically, FIG. 3 depicts a graph 301 of results or pharmacokinetic information for a pharmacokinetic model for predictions of a various etelcalcetide dosages and a graph 302 of corresponding clinical results from the Shen Study. Graphs 301 and 302 are configured as graphs of mean (+/−SD) of plasma intact etelcalcetide concentration versus time profiles for different etelcalcetide doses. A comparison of the pharmacokinetic information of graph 301 and the clinical results of graph 302 demonstrate that pharmacokinetic models according to some embodiments are able to simulate real-world clinical administration of calcimimetics, such as etelcalcetide, with high accuracy.

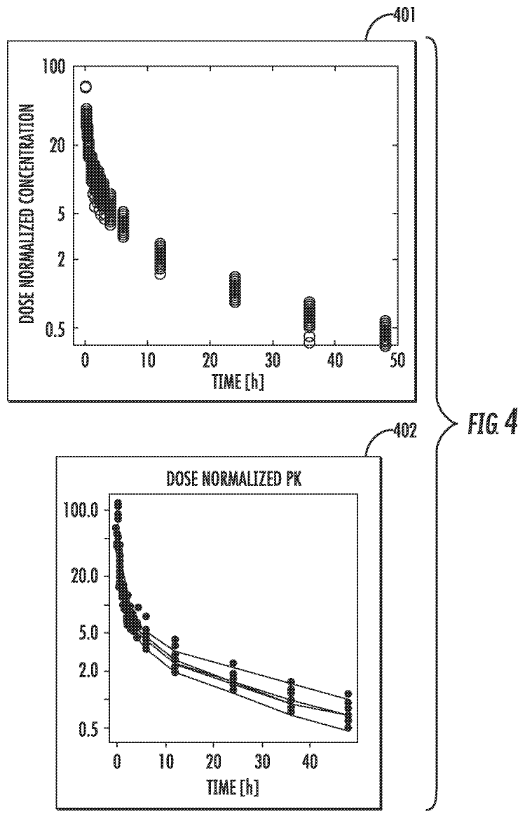

FIG. 4 depicts pharmacokinetics information for a pharmacokinetic model according to some embodiments. More specifically, FIG. 4 depicts a graph 401 of pharmacokinetic information for a pharmacokinetic model for a dose normalized pharmacokinetics profile versus time. Graph 402 depicts corresponding clinical results from the Shen Study. A comparison of the pharmacokinetic information of graph 401 and the clinical results of graph 402 demonstrate that pharmacokinetic models according to some embodiments are able to simulate real-world clinical administration of calcimimetics, such as etelcalcetide, with high accuracy.

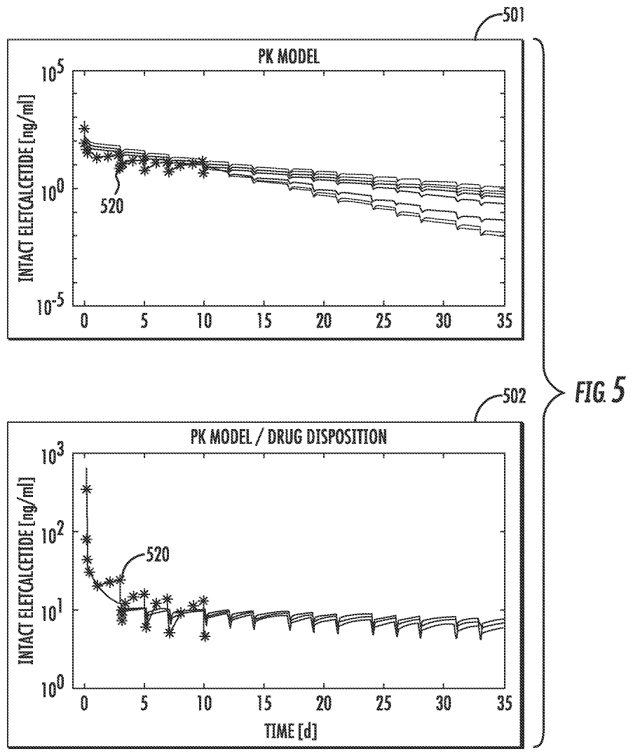

FIG. 5 depicts pharmacokinetics information for a pharmacokinetic model according to some embodiments. More specifically, FIG. 5 depicts a graph 501 of pharmacokinetic information for a pharmacokinetic model for a simulated single administration of 10 mg etelcalcetide in HD patients (N=100). The simulations are based on model parameters generated for healthy subjects with adjusted renal clearance and clearance during HD. The blood flow during dialysis as well as the hematocrit were random variables with a CV of 10%. Graph 502 depicts corresponding clinical results from Subramanian et al., “Nonclinical Pharmacokinetics, Disposition, and Drug-Drug Interaction Potential of a Novel D-Amino Acid Peptide Agonist of the Calcium-Sensing Receptor AMG 416 (Etelcalcetide),” Drug Metabolism and Disposition 44:1319-1331 (2016) (“Subramanian Study”). Elements 520 (i.e., star elements) display reference measurements presented in the Subramanian Study. A comparison of the pharmacokinetic information of graph 501 and the clinical results of graph 502 demonstrate that pharmacokinetic models according to some embodiments are able to simulate real-world clinical administration of calcimimetics, such as etelcalcetide, with high accuracy.

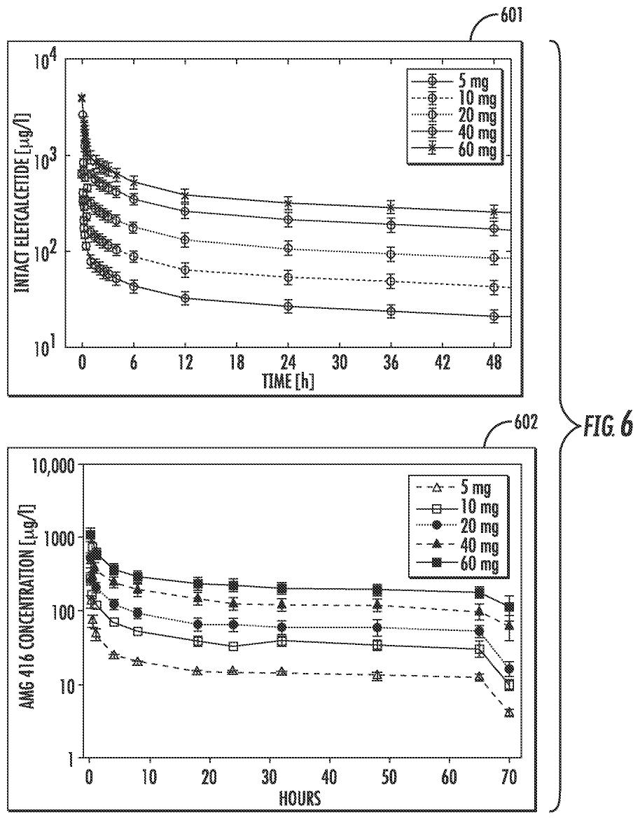

FIG. 6 depicts pharmacokinetics information for a pharmacokinetic model according to some embodiments. More specifically, FIG. 6 depicts a graph 601 of pharmacokinetic information in the form of pharmacokinetic profiles generated via a pharmacokinetic model for various dosages of etelcalcetide for HD patients (N=100). Graph 602 depicts corresponding clinical results from Martin et al., “AMG 416 (velcalcetide) is a novel peptide for the treatment of secondary hyperparathyroidism in a single-dose study in hemodialysis patients,” Kidney International, 85:191-197 (2014). A comparison of the pharmacokinetic information of graph 601 and the clinical results of graph 602 demonstrate that pharmacokinetic models according to some embodiments are able to simulate real-world clinical administration of calcimimetics, such as etelcalcetide, with high accuracy.

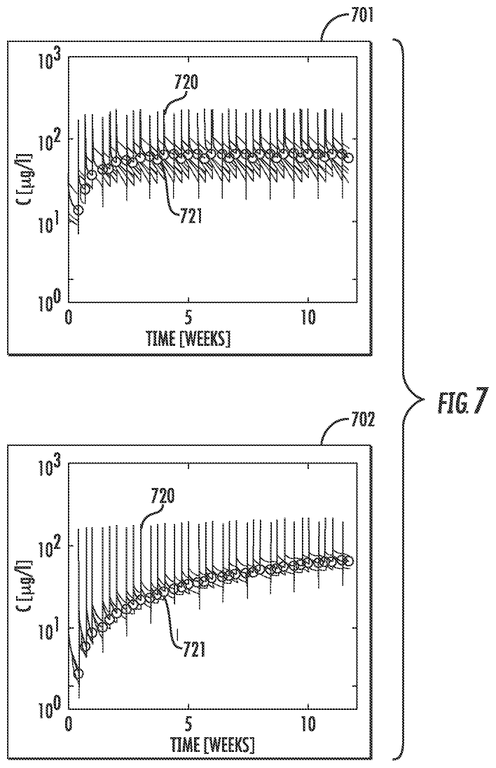

FIG. 7 depicts pharmacokinetics information for a pharmacokinetic model according to some embodiments. More specifically, FIG. 7 depicts a graph 701 of pharmacokinetic information generated via a pharmacokinetic model for administration of etelcalcetide to HD patients (N=10), for instance, with etelcalcetide administered after each HD session (for instance, 15 minutes after each HD session). Graph 702 depicts results from a simulation based on the drug disposition model (N=10). The trend line 720 depicts information for a median of all patients and circles 721 depict typical measurement times (for instance, 15 minutes after the start of the next HD session). As shown in graph 701, a steady state or plateau of etelcalcetide concentration is reached in patients at about 4 or 5 weeks. Clinical studies have indicated that etelcalcetide plasma concentration may reach a steady state at about 4 weeks (for instance, multiple 2.5 mg or 5 mg administrations of etelcalcetide after every HD session have been shown to reach a steady state around 4 weeks) and an accumulation ratio of between 2 and 3 fold. In some embodiments, a pharmacokinetic model may be configured for an accumulation ratio of about 3 fold. In various embodiments, a pharmacokinetic model may be configured for a steady state etelcalcetide concentration at about 4 weeks.

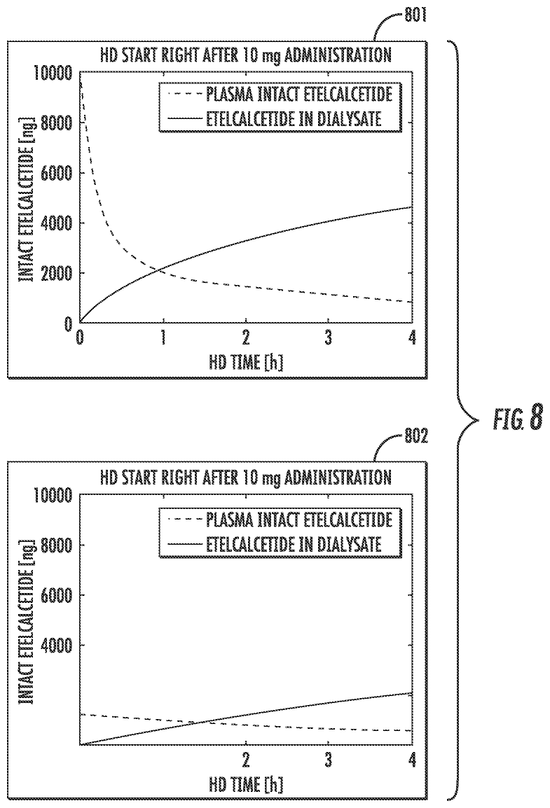

FIG. 8 depicts pharmacokinetics information for a pharmacokinetic model according to some embodiments. More specifically, FIG. 8 depicts a graph 801 of pharmacokinetic information generated via a pharmacokinetic model for administration of etelcalcetide before an HD session and graph 802 for administration of etelcalcetide 10 hours before an HD session. In general, graphs 801 and 802 may demonstrate the effect of biotransformation of etelcalcetide on the clearance by HD. More specifically, if etelcalcetide is administered before HD (graph 801), it is cleared swiftly by dialysis. If etelcalcetide is administered 10 hours before the start of hemodialysis (graph 802), a part of etelcalcetide is already biotransformed and not filterable via dialysis. The simulated results depicted in graphs 801 and 802 accurately predict corresponding data generated through in vitro clinical studies.

In some embodiments, a calcimimetic model may include a pharmacodynamic model configured to generate pharmacodynamic information. In some embodiments, pharmacodynamic information may include information associated with PTH, Ca, and/or phosphate in patients. In various embodiments, pharmacodynamic models may operate to predict a decrease (or other quantitative change) in PTH, Ca, and/or phosphate. In exemplary embodiments, pharmacodynamic models may predict efficacy endpoints for etelcalcetide administration, such as the fraction of patients with PTH reduction>30% (even for different PTH baseline strata), fraction of patients achieving PTH levels below a threshold (for instance, 300 pg/ml (even for different PTH baseline strata), mean phosphate, and ionized calcium reduction.

Clinical studies have established various pharmacodynamic properties of etelcalcetide. For example, etelcalcetide may lower intact PTH (iPTH) within 30 minutes after a single dose. A plateau in iPTH may be reached 1 hour post single dose administration. Serum ionized Ca (iCa) may be stable for 8 hours, but gradually decreases after about 18 hours; it may reach a plateau at 56 hours after a single dose administration. A decrease in phosphate may occur after about 4 hours post-administration and phosphate may remain low for about 4 days. In long term studies, iCa and phosphate declined within 12 weeks by around 10% and 5% before reaching a plateau. For HD patients, the iCa concentration may decline within 10 hours after administration of etelcalcetide in a dose-dependent manner and the highest changes in Ca concentration (around 15%) have been observed after a 60 mg administration.

Pharmacodynamic models according to some embodiments may include various configurations, assumptions, settings, parameters, and/or the like. For example, pharmacodynamic models may assume that CaSR exist in the two forms of active and inactive and the fraction of free receptors (L) is low. Ca and etelcalcetide bind to different sites on the CaSR and can discriminate between the two receptor states.

Pharmacodynamic models according to various embodiments may include the following transitions:

$$C + R + E {⇌}^{K} E + CR \\ C + R + E {⇌}^{M} RE + C \\ RE + C {⇌}^{γ K} CRE \\ E + CR {⇌}^{γ M} CRE \\ C + {R}^{*} + E {⇌}^{α K} E + C {R}^{*} \\ C + {R}^{*} + E {⇌}^{β M} {R}^{*} E + C \\ {R}^{*} E + C {⇌}^{αγδ K} {CR}^{*} E \\ E + C {R}^{*} {⇌}^{βγδ M} {CR}^{*} E \\ C + R + E {⇌}^{L} C + {R}^{*} + E \\ RE + C {⇌}^{β L} {R}^{*} E + C \\ CR + E {⇌}^{α L} {CR}^{*} + E \\ CRE {⇌}^{αβδ L} {CR}^{*} E$$

In various embodiments, the total concentrations of inactive receptors and active receptors may be determined according to the following:

[Rₜₒₜ]=[R]+[CR]+[RE]+[CRE]+[R*]+[CR*]+[R*E]+[CR*E] and

[R_{act}]=[R*]+[R*E]+[CR*]+[CR*E].

In some embodiments, dissociation constants may be used to express the fraction of active receptors according to the following:

$$\begin{matrix}\frac{[ {R}_{a c t} ]}{[ {R}_{t o t} ]} = \frac{[ {R}^{*} ] + [ C {R}^{*} ] + [ {R}^{*} E ] + [ C {R}^{*} E ]}{[ {R}_{t o t} ]} \\ = \frac{L ( 1 + α K [ C ] + β M [ E ] ( 1 + α γ δ K [ C ] )}{1 + L + M [ E ] ( 1 + β L ) + K [ C ] ( 1 + α L + γ M [ E ] ( 1 + α β δ L ) )}\end{matrix}$$

In the absence of etelcalcetide:

$$\frac{[ {R}_{a c t} ]}{[ {R}_{t o t} ]} = \frac{L ( 1 + α K [ C ] )}{1 + L + K [ C ] ( 1 + α L )}$$

The extreme values are:

$$\frac{[ {R}_{act} ]}{[ {R}_{t o t} ]} = \frac{L}{1 + L} for C = 0 \\ and \\ \frac{[ {R}_{act} ]}{[ {R}_{t o t} ]} {\to }_{C \to ∞} \frac{α L}{1 + α L} .$$

The constants may have the following meaning: α is the intrinsic efficacy of calcium, β is the intrinsic efficacy of etelcalcetide, γ is the binding cooperativity between calcium and etelcalcetide, δ is the activation cooperativity between calcium and etelcalcetide, K is the association constant of calcium, M is the association constant of etelcalcetide, and L is the receptor isomerization constant.

In one exemplary model, a is around 3.5, K=1/1.2, and L=0.05. If etelcalcetide had no effect on CaSR, β, γ, and δ would be equal 1. The effects of these parameters on the ln(calcium concentration)-active receptor curve are the following:
(a) If γ>1, the end-point and starting point of the curve are the same, the turning point of the s-shaped curve is shifted to lower calcium concentrations. γ<1 leads to a small shift of the turning point toward higher calcium concentrations;
(b) If β>1, starting and end point of the curve are increased. Since the increase in the end-point is more prominent, the slope of the curve is increased. β<1 has the opposite effect;
(c) If δ>1, the starting point of the curve is the same, the end-point is increased. Therefore, also the slope is increased. δ<1 has the same starting point, the end-point is decreased.

Pharmacodynamics models according to some embodiments may be based on the fraction of active receptors. For example, the fraction of the total receptor pool in the active state may be calculated, which determines the acute response of the PTG to CaSR activation.

The PTG biology model also takes the history of disturbances of mineral metabolism and calcitriol into account. Specifically, depending on the history of disturbances, PTH degradation rate is declined, PTH synthesis rate and cellular proliferation rate are elevated. Moreover, the carrying capacity of the parathyroid gland might be elevated resulting in hyperplasia. To take care of the effect of etelcalcetide on the overall PTG biology, embodiments may use the following effect compartment for the CaSR:

$$\frac{{dC}_{sens}}{dt} = {τ}_{sens} \cdot {( C \cdot \frac{ρ}{ρ 0} - {C}_{sens} )}_{n}$$

where C is the ionized calcium concentration, Cₛₑₙₛ is the effective calcium concentration associated with the fractional CaSR occupancy (ρ), and ρ₀ is the fraction CaSR occupancy before the start with etelcalcetide. The effective calcium concentration may determines PTG biology, for instance, the changes in intracellular degradation rate, synthesis rate, and cellular proliferation rate, and/or the like. Moreover, it has been shown that calcimimetics effectively enhance PTG apoptosis rate, thereby reducing the size of hyperplastic glands. This characteristic may be managed by reducing the apoptosis rate as well as the growing capacity via the following stimulus function depending on etelcalcetide concentrations:

$$\frac{d {S}_{e t e}}{d t} = {k}_{e t e} sete \cdot {S}_{e t e} + {r}_{e t e} \cdot ( 1 - {S}_{E t e} ) \\ \frac{d {s}_{e t e}}{d t} = ( s t i {m}_{e t e} \cdot ( 1 - \frac{s t i {m}_{e t e}}{\vert sti {m}_{e t e} \vert} {s}_{e t e} ) - {s}_{e t e} ) {τ}_{e t e} \\ {stim}_{e t e} = L \cdot ( \frac{1}{1 + exp ( - {k}_{1} \cdot ( C - {C}_{1} ) )} + \frac{1}{1 + exp ( - {k}_{1} \cdot ( C - {C}_{2} ) )} )$$

In various embodiments, the pharmacodynamic model may be linked with the pharmacokinetics model by introducing an effect compartment which equilibrates with etelcalcetide plasma concentration with time, for example, via the following:

$$\frac{d {E}_{e}}{d t} = {k}_{eff} \cdot ( {E}_{p} - {E}_{e} ) ,$$

where Eₑ is the amount if etelcalcetide in the effect compartment, for instance, the parathyroid gland surface. In addition, a negative feedback loop may be used between plasma ionized calcium concentration (C) and phosphate (P):

$$\frac{d C}{d t} = {K}_{i n , C} \cdot ( 1 + {s}_{C} ( P T H - P T {H}_{0} ) ) - {K}_{out , C} \cdot C \\ \frac{d P}{d t} = {K}_{i n , P} \cdot ( 1 + {s}_{P} ( P T H - P T {H}_{0} ) ) - {K}_{out , P} \cdot P .$$

In healthy subjects, s_{P} would be negative due to the phosphaturic effect of PTH. In HD patients, both constants, s_{Ca} and s_{P}, are positive.

Single etelcalcetide administration in healthy patients may be simulated using calcimimetic models configured according to some embodiments. The following Table 2 provides a parameter set for simulating etelcalcetide administration in healthy patients:

| Parameter | Estimate | |
|---|---|---|
| K_{out, C} | 0.0232 | l/h |
| K_{out, P} | 0.05 | l/h |
| S_{C} | 0.06 | mMol/pMol |
| S_{P} | −0.2 | mMol/pMol |
| γ | 1.1 | 1/h |
| β | 1.8 | 1/h |
| δ | 1.1 | 1/h |
| M | 1/600 | L/μg |

In some embodiments, the parameter keff=0.3, for example, in order to ensure maximum PTH suppression at around 30 minutes after administration.

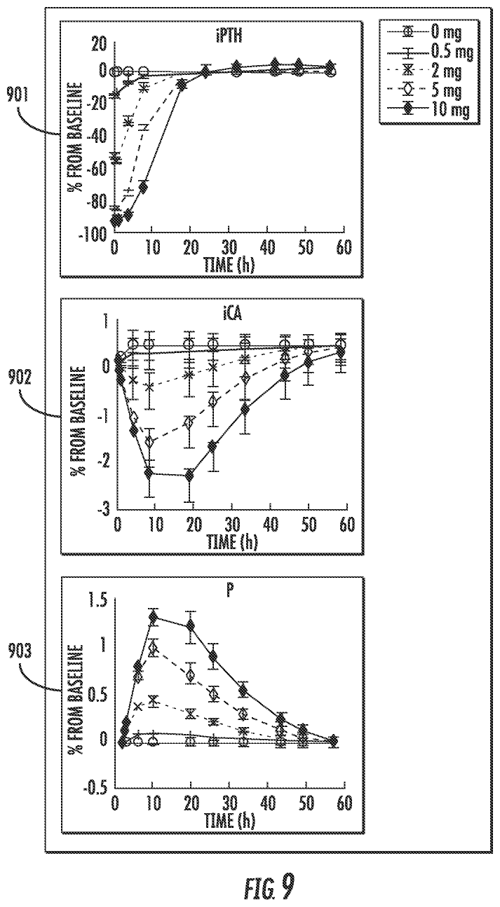

FIG. 9 depicts model information for simulating etelcalcetide administration in healthy patients according to some embodiments. As shown in FIG. 9, graphs 901-903 depict results of the pharmacodynamics and pharmacokinetics models in healthy subjects (N=10). The longer lasting effect of etelcalcetide in PTH suppression can be reached by adjusting the pharmacodynamics parameters and/or adjusting the slope in the Ca/PTH release rate function. However, with the set of parameters of Table 2 a single etelcalcetide administration in HD patients was able to be simulated. Five different doses (placebo, 0.5 mg 2 mg, 5 mg, and 10 mg) of single etelcalcetide administrations were simulated in healthy subjects. Gaussian noise was added to the optimal PD parameters (1% CV).

In some embodiments, the parameters of the pharmacodynamics model may not differ between healthy subjects and HD patients. At least one difference may be calcium and phosphate response. Since bone and mineral metabolisms is altered in HD patients, it may be assumed that calcium reacts faster to changes in PTH compared to healthy subjects, for example, based at least on high bone turnover in HD patients. Therefore, a decline in PTH may trigger a decline in Ca much faster in HD patients compared to healthy subjects with normal bone turnover and Ca and phosphate homeostasis. Moreover, without residual kidney function, phosphate is supposed to decline if PTH declines. Accordingly, some embodiments may operate using the following configurations: K_{out,C}=0.16 l/h, K_{out,P}=0.025 l/h, s_{C}=s_{P}=0.004 mMol/pMol.

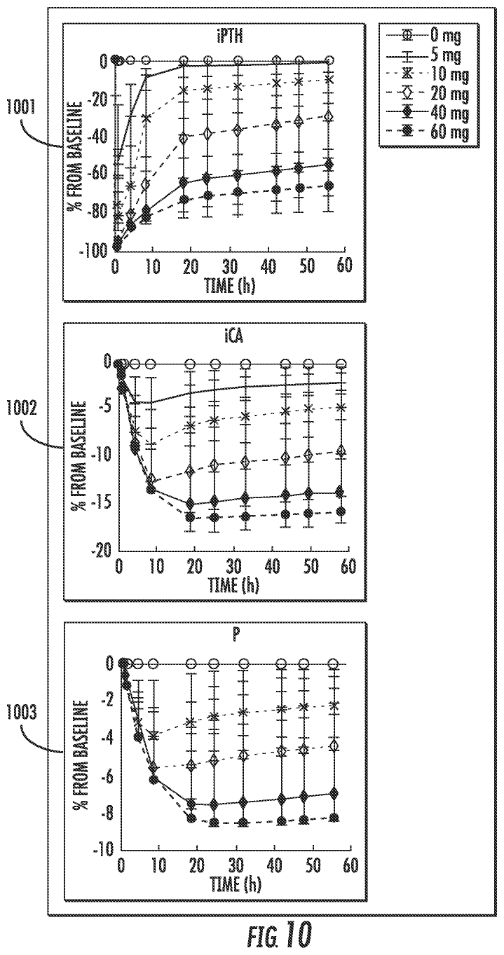

FIG. 10 depicts model information for simulating etelcalcetide administration in HD patients according to some embodiments. As shown in FIG. 10, graphs 1001-1003 depict results of the pharmacodynamics and pharmacokinetics models in healthy subjects (N=10). Due to the strong suppression, 20-60 mg show almost the same effect. The parameters of the pharmacokinetics model may be based on the boot strap method. Gaussian noise may be added to the optimal pharmacodynamics parameter set (1% CV).

Multiple etelcalcetide administration in patients may be simulated using calcimimetic models configured according to some embodiments. For HD patients, the following are non-limiting model configurations: every HD session may last 4 hours; etelcalcetide may be administered after the end of an HD session; PTH may be measured before the start of an HD session; and etelcalcetide may be dose titrated.

The strong lowering effects of etelcalcetide may result in hypocalcemia. In clinical studies, around 7.6% of the patients had at least one corrected serum calcium values below 7 mg/dl, 27% had at least one corrected serum calcium value below 7.5 ng/dl, and 79% had at least one corrected calcium value below 8.3 mg/dl. Therefore, in some patients, dose titration is essential in order to avoid severe hypocalcemia. Based on the prescribing information, the following are non-limiting examples of model configurations that may be used to simulate dose titration:
The dose may be adjusted on a constant time span, such as every 4 weeks. In some embodiments, the dose may be adjusted on the same day of the week;
If iCa is below 0.9375 mMol (which should translate to a corrected calcium concentration of 7.5 mg/dl), etelcalcetide administration may be put on hold until iCa has recovered and is greater than 1.03 mMol (which should translate to a corrected serum calcium concentration of about 8.3 mg/dl);
If iPTH is below 100 pg/ml and/or iCa is below 1.06 mMol, the etelcalcetide dose may be reduced, for instance, by 2.5 mg;
If iPTH is between 100 and 600 pg/ml, the etelcalcetide dose may be unchanged. However, if etelcalcetide was on hold before, etelcalcetide is re-initiated with a dose 5 mg lower than the last administered dose. If the last administered dose was 2.5 mg or 5 mg, the re-initiation dose is 2.5 mg;
If iPTH is between 300 and 600 pg/ml, the etelcalcetide dose is raised, for example, by 2.5 mg. However, if etelcalcetide was on hold before, etelcalcetide is re-initiated with a dose 5 mg lower than the last administered dose. If the last administered dose was 2.5 mg or 5 mg, the re-initiate dose is 2.5 mg;
If iPTH is above 600 pg/ml, the etelcalcetide dose is raised, for example, by 5 mg. However, if etelcalcetide was on hold before, etelcalcetide is re-initiated with a dose 5 mg lower than the last administered dose. If the last administered dose was 2.5 mg or 5 mg, the re-initiate dose is 2.5 mg; and

The model may use a maximum dose, for example, of 15 mg.

Certain other configurations may be applied for simulations based on different clinical studies. For example, dose titration may only be allowed during certain weeks, such as only being allowed in weeks 5, 9, 13, and/or 17. In another example, etelcalcetide may be withheld if PTH is less than 100 pg/ml and serum Ca is less than 7.5 mg/dl.

A pool or population of patients may be obtained or generated according to some embodiments. The patient population may be used in simulations of calcimimetic models according to various embodiments. In exemplary embodiments, the patient population may be created based on the generated reference values for the pharmacokinetic model and one or more of the following configurations for the PTG biology model:
The PTG is in a pseudo-steady state before the start with etelcalcetide, for example, without changes in phosphate, calcium and calcitriol, the state of the PTG and PTH release will not change significantly within a small time frame;
Individual phosphate concentrations [mmol/l] at the start of etelcalcetide administration are normally distributed with mean 1.9 mmol/l and CV of 5%;
Patients with high phosphate levels seem to have higher calcium levels. Moreover, high phosphate levels increase PTG proliferation rate. Therefore, individual Ca concentrations [mmol/l], phosphate and growing capacity have a common distribution. Mean ionized Ca concentration is 1.19 mmol/l, the CV of the common distribution is 5%. In addition, Gaussian noise may be added (0.1% CV) to ionized Ca concentration and growing capacity;
Individual calcitriol concentrations [pmol/l] at the start of etelcalcetide administration are normally distributed with mean 45 pmol/l and Cv of 10%; and/or
The individual PTG parameters intracellular degradation rate (k_{d}), synthesis rate (kₚ), clearance rate (k_{cl}), proliferation rate (kₚₗ), and growing capacity are normally distributed.

The following Table 3 provides mean values and CV of PTG model parameters that may be used in calcimimetic model simulations according to some embodiments:

TABLE 3

| Parameter | Mean Value | CV | Healthy gland |
|---|---|---|---|
| r_{d} | 0.36 | 10% | 0.72 |
| kₚ | 7920 | 50% | 3960 |
| k_{cl} | 28.338 | 10% | 56.676 |
| kₚₗ | 2.16 | 10% | 1.8 |
| K | 3.7 | 30% | 1 |

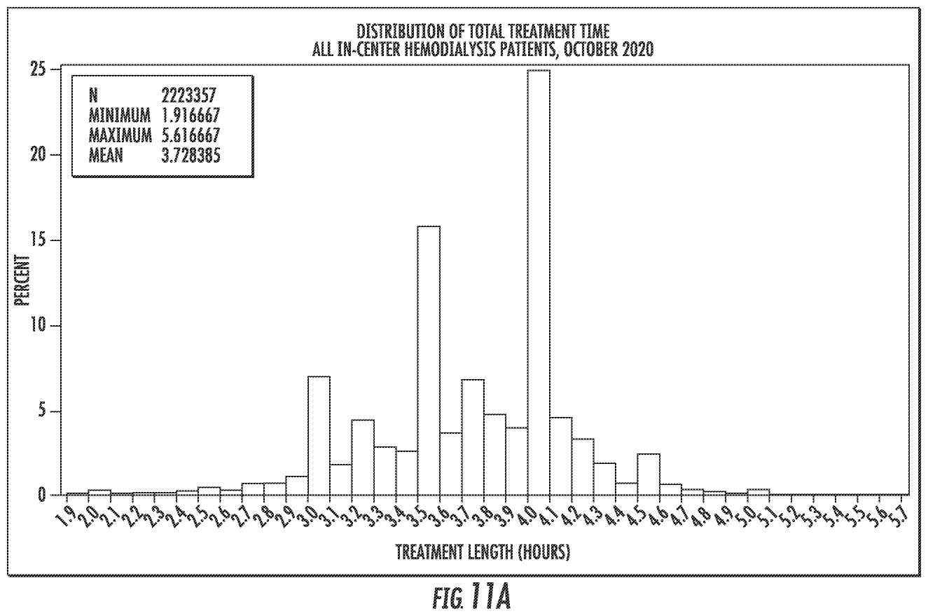

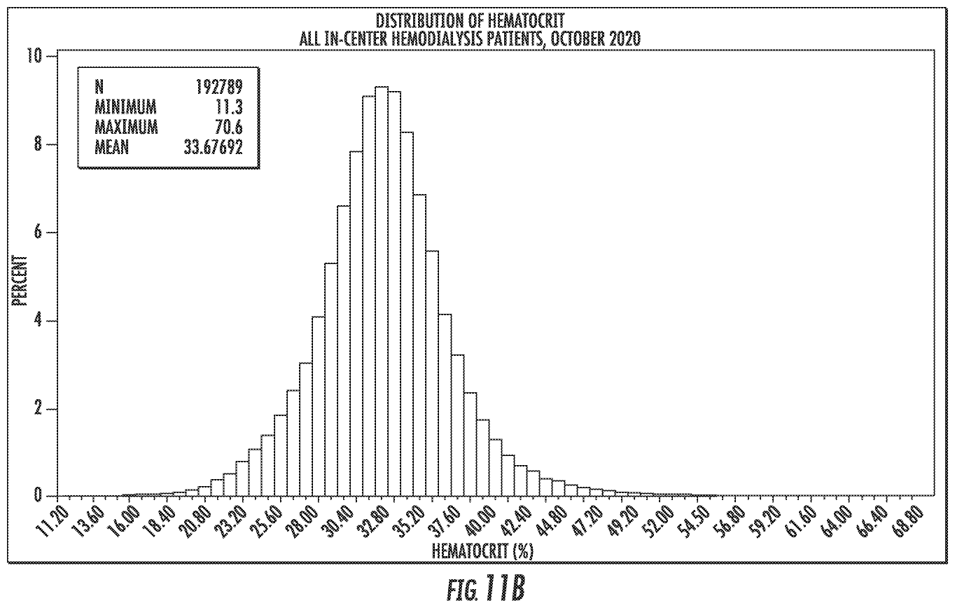

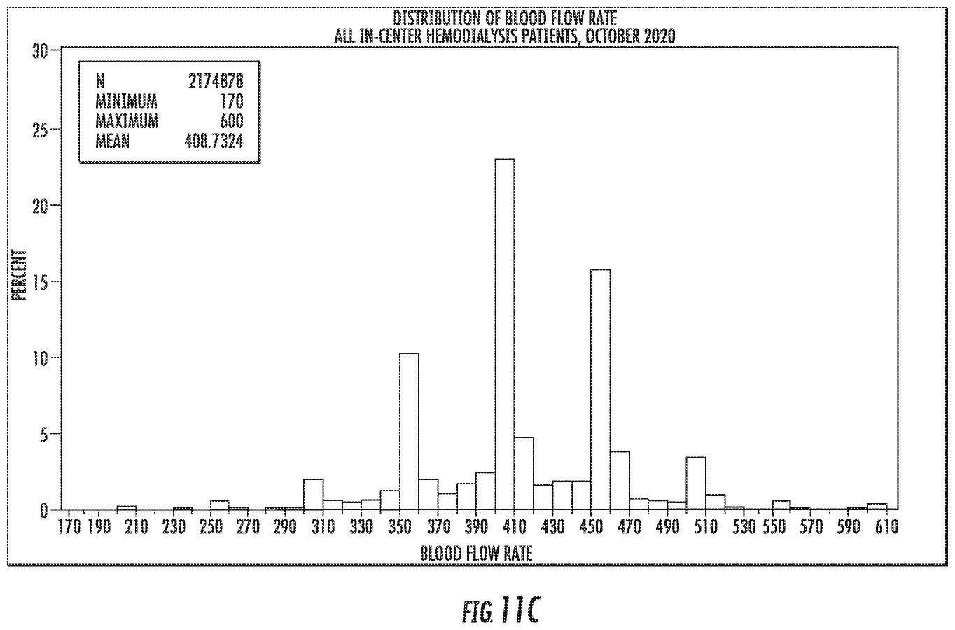

FIGS. 11A-11C depict illustrative HD information for in-center HD patients over a one-month time span. More specifically, FIGS. 11A-11C depict graphs of the distribution of total treatment time, the distribution of hematocrit, and the distribution of blood flow rate, respectively, for Fresenius Medical Care (FMC) (or Fresenius Kidney Care) in-center HD patients during October 2020.

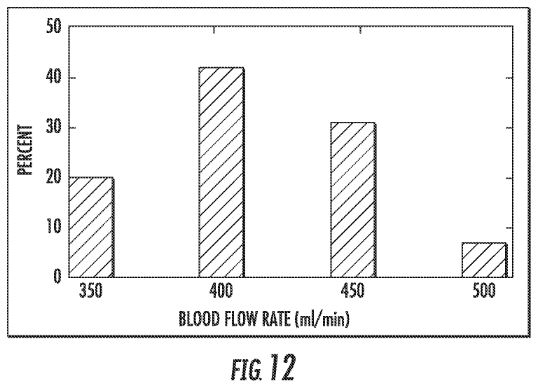

Based on the distributions provided in FIGS. 11A-11C, calcimimetic model simulations may randomly draw a value from the distribution of the hematocrit and dialyzer blood flow rate for a discretization blood flow distribution for a FMC patient population simulation. FIG. 12 depicts an illustrative and non-limiting example of a discretization blood flow distribution according to some embodiments. In some embodiments, calcimimetic models may generate simulations using the standard four-hour treatment length to compare the results with the clinical results, as well as simulated treatment lengths of 3.5 hours and 3 hours, for example, to determine the effect of treatment length on the PTH, iCa, and phosphate predictions. Since etelcalcetide is cleared mainly be HD, a shorter treatment length may result in a higher suppression of PTH.

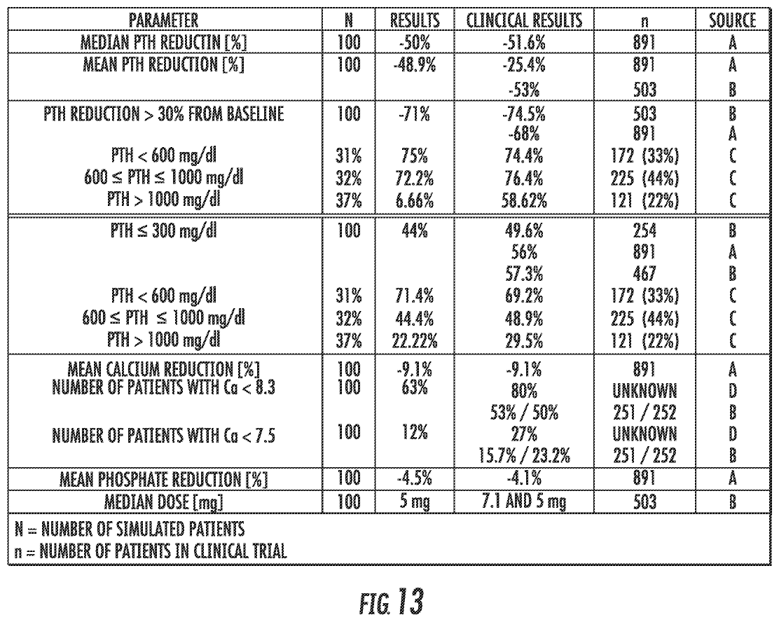

FIG. 13 depicts calcimimetic model information according to some embodiments. More specifically, FIG. 13 includes Table 1301 providing a comparison of simulation results generated via calcimimetic models according to some embodiments and the efficacy phase of the following various clinical studies: Source A: Bushinsky et al., “One Year Efficacy and Safety of Intravenous (IV) Etelcalcetide (AMG 416) in Patients on Hemodialysis (HD) with Secondary Hyperparathyroidism (SHPT),” Nephrology Dialysis Transplantation, 31:13-14 (2016); Source B: Block et al., “Cinacalcet for Secondary Hyperparathyroidism in Patients Receiving Hemodialysis,” New England Journal of Medicine, 350:1516-1525 (2004); Source C: Cunningham et al., “Etelcalcetide Is Effective at All Levels of Severity of Secondary Hyperparathyroidism in Hemodialysis Patients,” Kidney International Reports, 4:987-994 (2019); and Source D: Amgen, “Highlights of Prescribing Information” for PARSABI (etelcalcetide) injection, for intravenous use, Initial U.S. Approval: 2017.

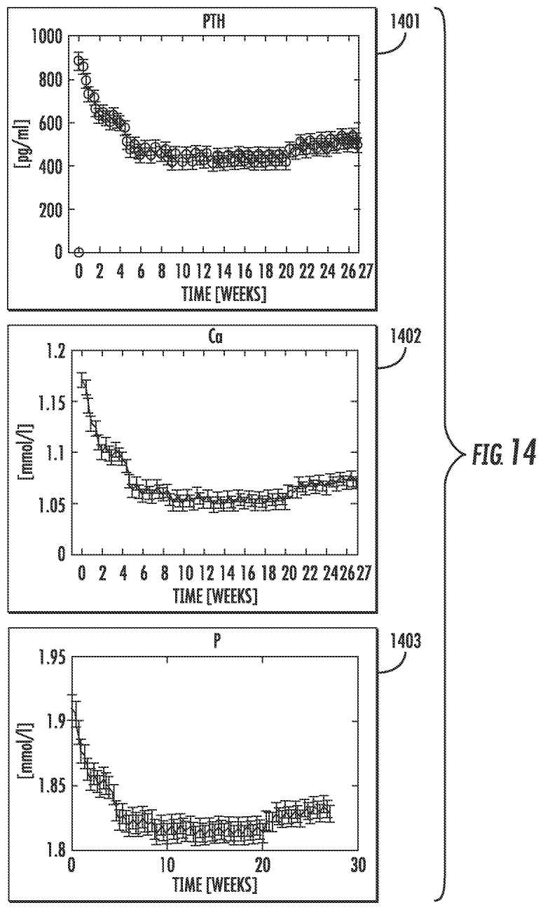

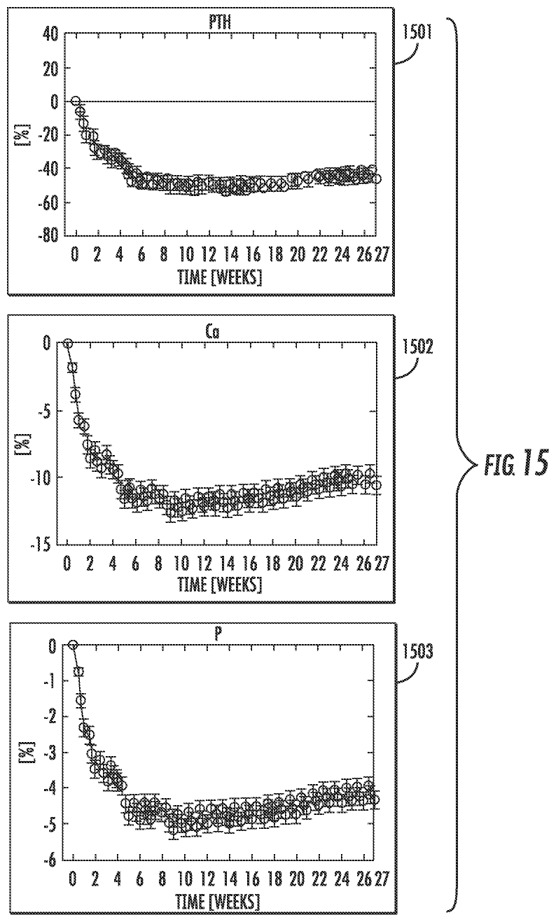

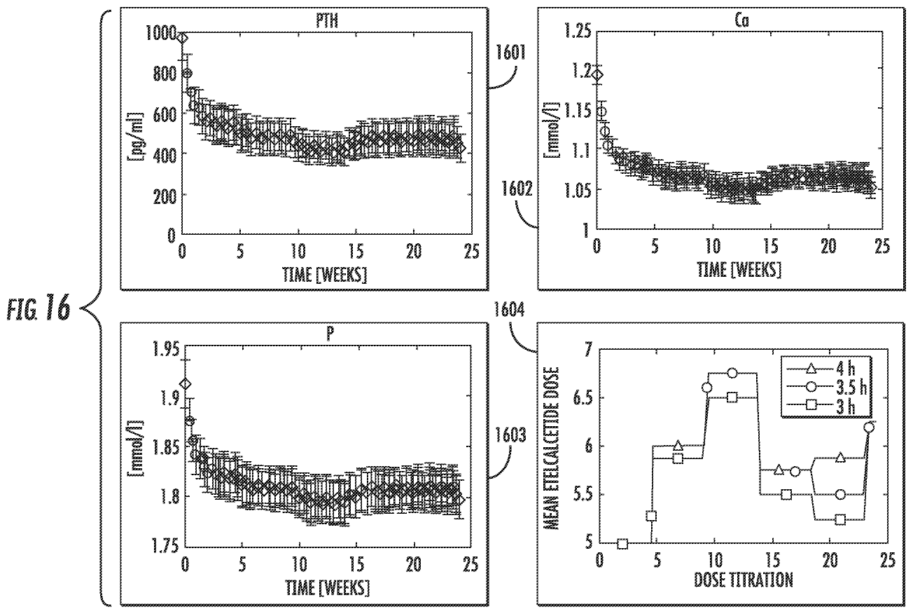

FIGS. 14-16 depict calcimimetic information generated via calcimimetic models according to some embodiments. More specifically, FIG. 14 depicts graphs 1401-1403 for PTH, Ca, and phosphate based on simulations generated via pharmacodynamic models and pharmacokinetic models according to various embodiments, using the FMC patient population with N=100 and with results presented as mean (+/−SEM). As indicated in FIG. 14, Ca may recover slightly around week 12 due to dose titration. FIG. 15 depicts graphs 1501-1503 for PTH, Ca, and phosphate based on simulations generated via pharmacodynamic models and pharmacokinetic models according to various embodiments with N=100 and with results presented as mean (+/−SEM). The results are in close accordance with the clinical trial data of Source A and Source B of FIG. 13. FIG. 16 depicts graphs 1601-1603 for PTH, Ca, and phosphate and a dose titration scheme 1604 for a patient population of N=20. As indicated in FIG. 16, treatment length may not have an effect on PTH reduction.

In some embodiments, calcimimetic models may be configured for alternative administration schemes or processes. In general, based on a conventional or classical dose titration scheme (e.g., the dose can be adjusted every 4 weeks; doses can be adjusted in 2.5 mg steps; the target iPTH range may be between 150 and 600 pg/ml; if Ca_{corr} falls below 8 mg/dl, etelcalcetide may be put on hold until Ca_{corr} is higher than a threshold value, such as 8.5 mg/dl.), twice weekly may be demonstrated to be non-inferior in controlling PTH to thrice weekly. This is mainly due to the drop in ionized calcium concentrations which makes pausing and restarting of etelcalcetide necessary in the thrice weekly administration. Based on the classical dose titration scheme, once weekly seems to be able to control PTH not as well as thrice weekly in patients with high PTH baseline values (>800 pg/ml). As described in more detail in the present disclosure, simulation results included a higher number of simulated patients and detailed characterization of patients where thrice weekly is more successful than once or twice weekly.

In clinical trials, a titration scheme may be based on trying to achieve a particular iPTH value, such as iPTH≤300 pg/ml. A titration process according to some embodiments may include one or more of the following titration scheme characteristics:
The dose can be adjusted every 4 weeks;
The target PTH range is 150-600 pg/ml;
If PTH<150 pg/ml, etelcalcetide administration is on hold until PTH>150 pg/ml;
If Ca_{Corr}<8 mg/dl, etelcalcetide administration is on hold until Ca_{Corr}≥8.5 mg/dl;
If iPTH is between 151 and 400 pg/ml, the dose is decreased by 2.5 mg;
If iPTH is between 400 and 800 pg/ml and the difference in 4-weeks iPTH>100 pg/ml, the dose is decreased by 2.5 mg;
If iPTH is above 800 pg/ml, the dose is increased by 2.5 mg;
The maximum dose is 15 mg;
Re-initiation: the dose is either 5 mg lower than the last administered dose or 2.5 mg—whichever dose is larger; and

The target range for calcium is 8.5-10 mg/dl, the target range for phosphate is 3-5 mg/dl.

Some embodiments may operate to simulate alternative administration processes. For example, simulation using calcimimetic models according to some embodiments may be based on a large number of patients, such as 1000 patients, with 100 randomly chosen patients per an iPTH baseline stratum:
300 pg/ml≤PTH baseline<500 pg/ml;
500 pg/ml≤PTH baseline<800 pg/ml;
800 pg/ml≤PTH baseline<1000 pg/ml; and
PTH baseline>1000 pg/ml.

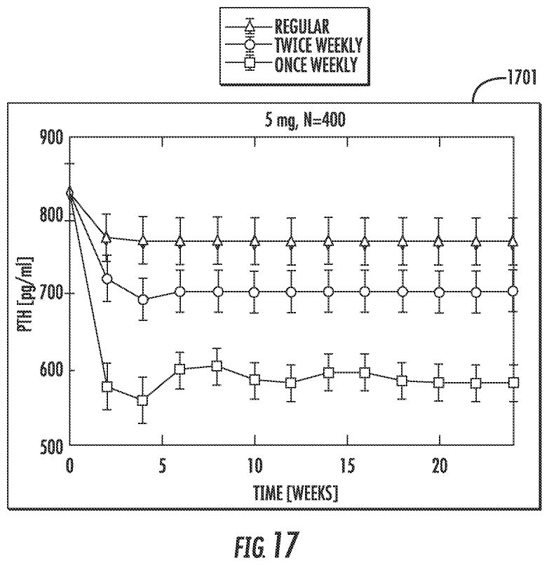

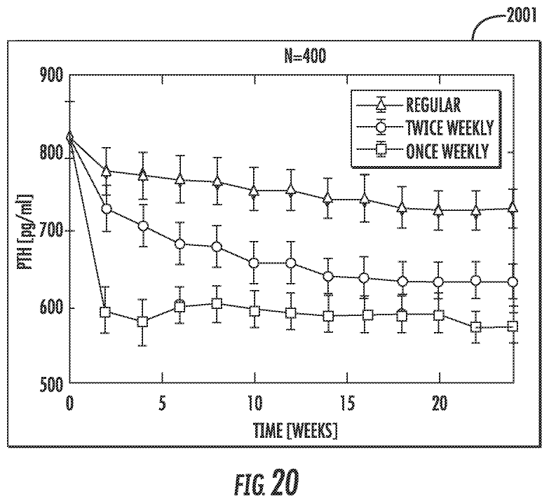

In some embodiments, for example, simulations may use the same patients to simulate regular administration as well as twice weekly and once weekly administration. In addition, simulations may be performed in which a maximum of 5 mg was administered twice or once weekly without the option of up-titrating the dose. In general, such simulations may show, among other things, the efficacy of once or twice weekly based on the lowest etelcalcetide dose. Summaries of the simulation results are provided in the following Table 4 (see also, FIGS. 17-19) and Table 5 (see also, FIGS. 20-22):

TABLE 4

| Parameter | Regular | Twice weekly | Once weekly |
|---|---|---|---|
| PTH (150-600 pg/ml) | 55% | 49% | 41% |
| Ca (8.5-10 mg/dl) | 54% | 62% | 62% |
| Phosphate (3-5.5 mg/dl) | 44.3% | 32.5% | 19.5% |
| Mean total dose [mg] | 368 mg | 215 mg | 115 mg |
| Mean number of administration | 66.4 | 47 | 23.1 |
| Mean dose/administration | 5.5 mg | 4.6 mg | 4.9 mg |

TABLE 5

| Parameter | Regular | Twice weekly | Once weekly |
|---|---|---|---|
| PTH (150-600 pg/ml) | 60% | 61% | 46.5% |
| Ca (8.5-10 mg/dl) | 54.3% | 53.8% | 62.8% |
| Phosphate (3-5.5 mg/dl) | 41.8% | 40% | 26.8% |
| Mean total dose [mg] | 397 mg | 323.5 mg | 180.8 mg |
| Mean number of administration | 66.9 | 47 | 23.2 |
| Mean dose/administration | 5.8 mg | 6.6 mg | 7.7 mg |

Table 4 provides calcimimetic information for simulations using calcimimetic models according to some embodiments for patients within PTH, phosphate, and iCa target range for thrice weekly, twice weekly, and once weekly administration. In the simulations for Table 4, no up-titration was allowed in the twice weekly and once weekly administration processes. In the simulations for Table 5, up-titration was allowed in the twice weekly and once weekly administration processes.

FIG. 17 depicts simulation results for an illustrative calcimimetic model according to some embodiments. More specifically, FIG. 17 depicts graph 1701 of pharmacodynamics information in the form of PTH concentration versus time. In general, graph 1701 may demonstrate, among other things, the effect of different administration schemes on PTH reduction. In the simulation corresponding to FIG. 17, up-titration was not allowed in the twice weekly or once weekly administration schemes.

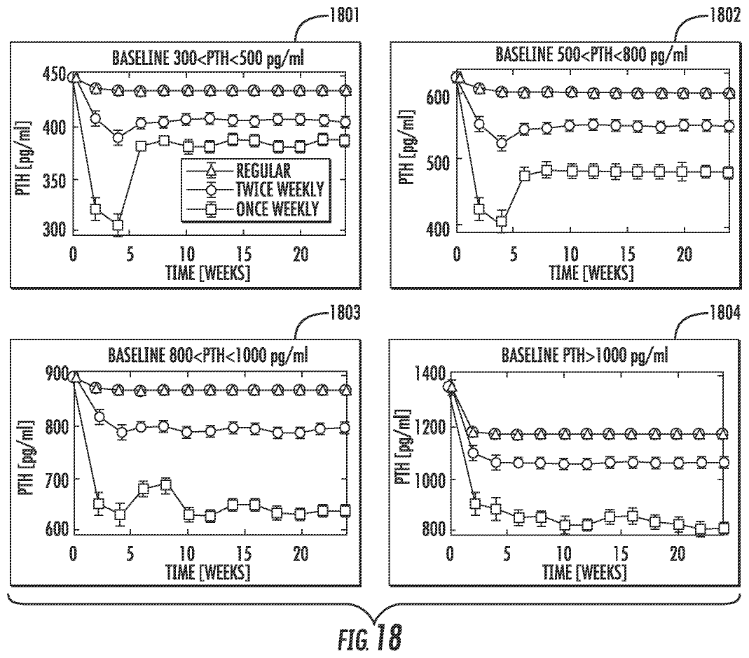

FIG. 18 depicts simulation results for an illustrative calcimimetic model according to some embodiments. More specifically, FIG. 18 depicts graphs 1801-1804 of pharmacodynamics information in the form of PTH concentration versus time for various PTH baselines. In the simulation associated with FIG. 18, up-titration was not allowed in the twice weekly or once weekly administration scheme. The corresponding average single doses for the four PTH baseline strata are (300-500 pg/ml (1801), 500-800 pg/ml (1802), 800-1000 pg/ml (1803), >1000 pg/ml (1804)): 3.9 mg, 4.3 mg, 6.2 mg, and 7.5 mg in the thrice weekly group; 4.1 mg, 4.6 mg, 4.9 mg, and 4.8 mg in the twice weekly group; 4.8 mg, 5 mg, 4.9 mg, and 5 mg in the once weekly group.

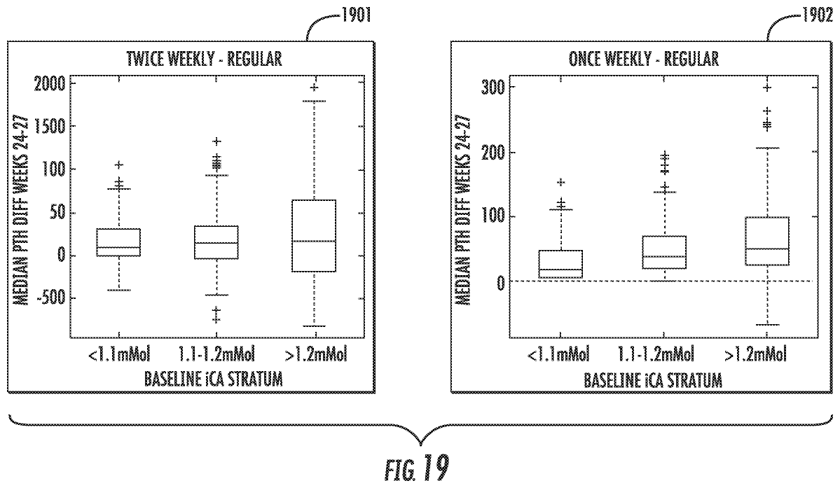

FIG. 19 depicts simulation results for an illustrative calcimimetic model according to some embodiments. More specifically, FIG. 19 depicts graphs 1901 and 1902 of median of mean differences between twice weekly (1901) and once weekly (1902) regular iPTH concentrations in the efficacy phase (for instance, weeks 24-27). In the simulation corresponding to FIG. 19, no up-titration was allowed. In general, higher iCa baseline levels allow greater up-titration in conventional or regular titration schemes; therefore, differences may be greatest at high baseline iCa concentrations.

FIG. 20 depicts simulation results for an illustrative calcimimetic model according to some embodiments. More specifically, FIG. 20 depicts graph 2001 of pharmacodynamics information in the form of PTH concentration versus time. In general, graph 2001 may demonstrate, among other things, the effect of different administration schemes on PTH reduction. In the simulation corresponding to FIG. 20, up-titration was allowed in the twice weekly or once weekly administration schemes.

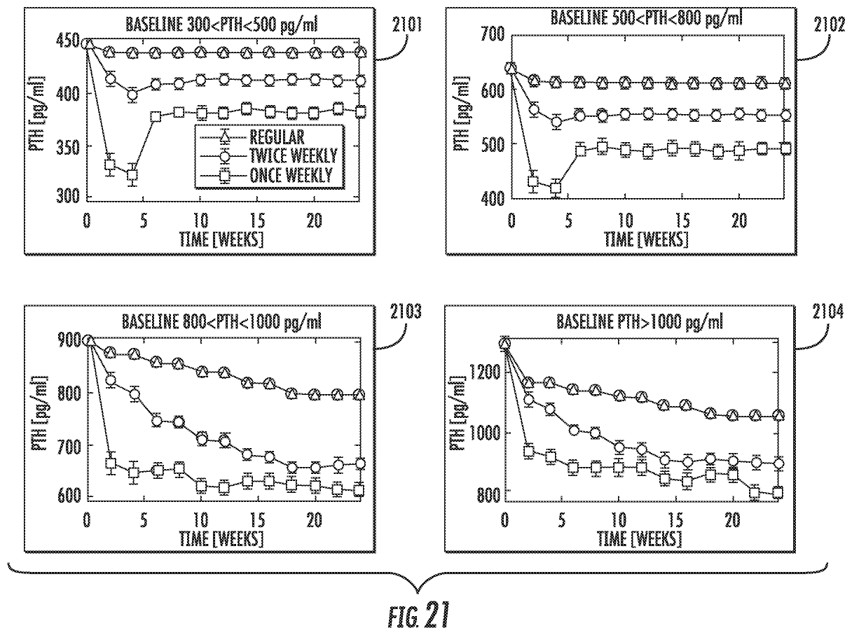

FIG. 21 depicts simulation results for an illustrative calcimimetic model according to some embodiments. More specifically, FIG. 21 depicts graphs 2101-2104 of pharmacodynamics information in the form of PTH concentration versus time for various PTH baselines. In the simulation associated with FIG. 21, up-titration was allowed in the twice weekly or once weekly administration scheme. The corresponding average single doses for the four PTH baseline strata are (300-500 pg/ml (1801), 500-800 pg/ml (1802), 800-1000 pg/ml (1803), >1000 pg/ml (1804)): 4.3 mg, 4.2 mg, 6.7 mg, and 8.1 mg in the thrice weekly group; 4.2 mg, 4.8 mg, 8.9 mg, and 9.5 mg in the twice weekly group; 4.8 mg, 5 mg, 10.4 mg, and 10.5 mg in the once weekly group.

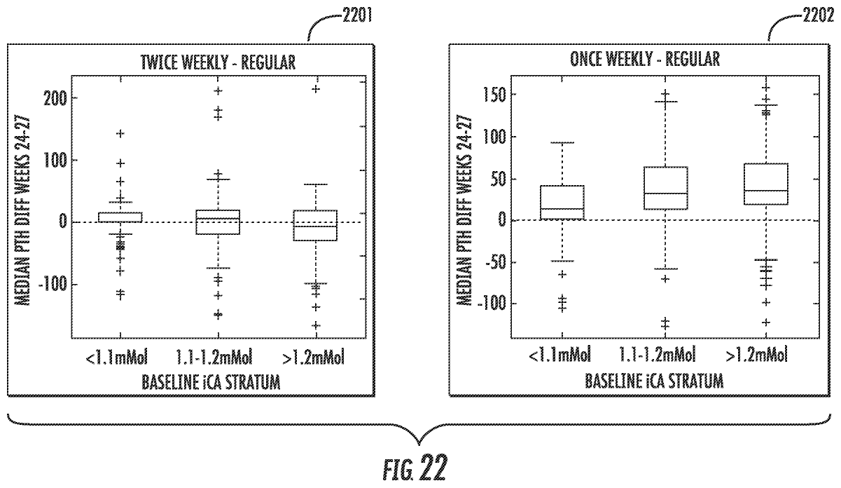

FIG. 22 depicts simulation results for an illustrative calcimimetic model according to some embodiments. More specifically, FIG. 22 depicts graphs 2201 and 2202 of median of mean differences between twice weekly (2201) and once weekly (2202) regular iPTH concentrations in the efficacy phase (for instance, weeks 24-27). In the simulation corresponding to FIG. 22, up-titration was allowed in the once weekly and twice weekly administration schemes.

Various biological compounds may influence the efficacy of etelcalcetide. For example, phosphate may have an influence on etelcalcetide efficacy. Accordingly, calcimimetic models may include phosphate-adjusted models configured to adjust for the effects of phosphate. For instance, a calcimimetic model may adjust for the direct effect of phosphate on CaSR activation. In another example, high phosphate baseline values may lower the probability of achieving a PTH target range, irrespective of serum Ca and PTG growth capacity.

In various embodiments, calcimimetic models may include a PTG model adjusted to accommodate the immediate effect of phosphate on PTH release. Phosphate may act directly on the CaSR via non-competitive antagonism. Specifically, phosphate reduces the maximal response of the CaSR to Ca²⁺ but does not alter Ca²⁺ potency. Accordingly, the maximal release rate A may be adjusted based on phosphate concentrations according to the following:

$${R}_{p h o s} = {α}_{P h o s} + ( {β}_{P h o s} - {α}_{P h o s} ) \cdot \frac{{P}^{{γ}_{Phos}}}{{P}^{{γ}_{Phos}} + {K}_{P}^{{γ}_{P h o s}}} \\ A = A \cdot {R}_{phos}$$

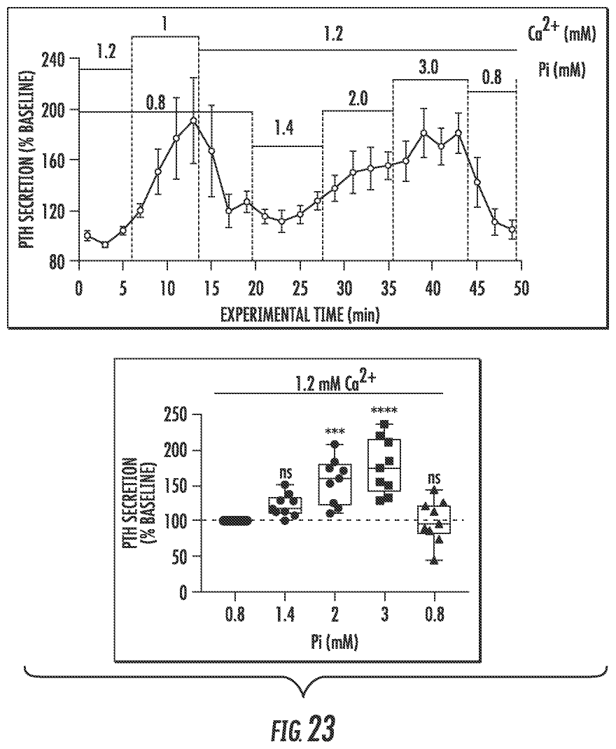

The model may be calibrated with the experimental results. FIG. 23 depicts experimental results used to calibrate a phosphate-adjusted calcimimetic model.

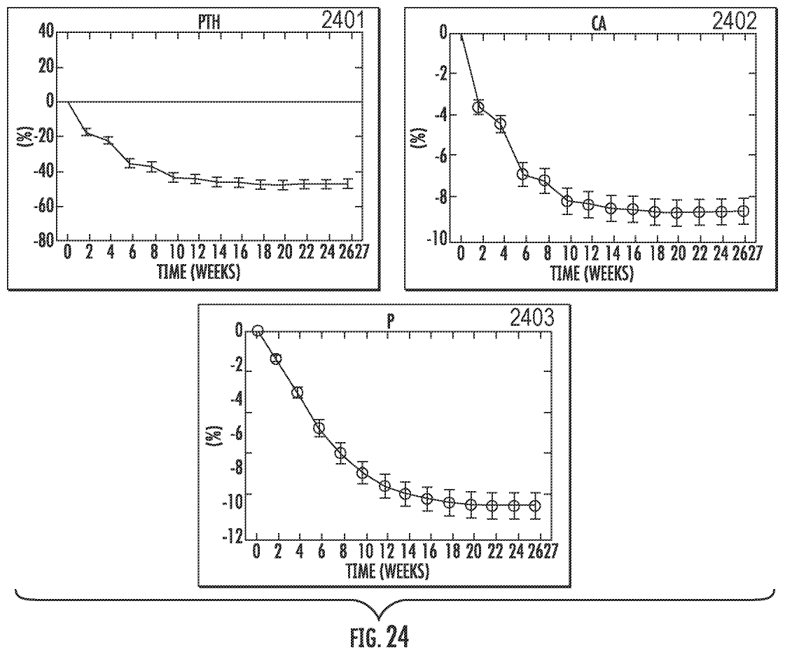

FIG. 24 depicts calcimimetic information generated via a phosphate-adjusted calcimimetic model according to some embodiments. More specifically, FIG. 24 depicts graphs 2401-2403 for PTH, Ca, and phosphate based on simulations generated via phosphate-adjusted calcimimetic model according to various embodiments, using a patient population with N=100. The results are in close accordance with published clinical data, including Source A and Source B of FIG. 13.

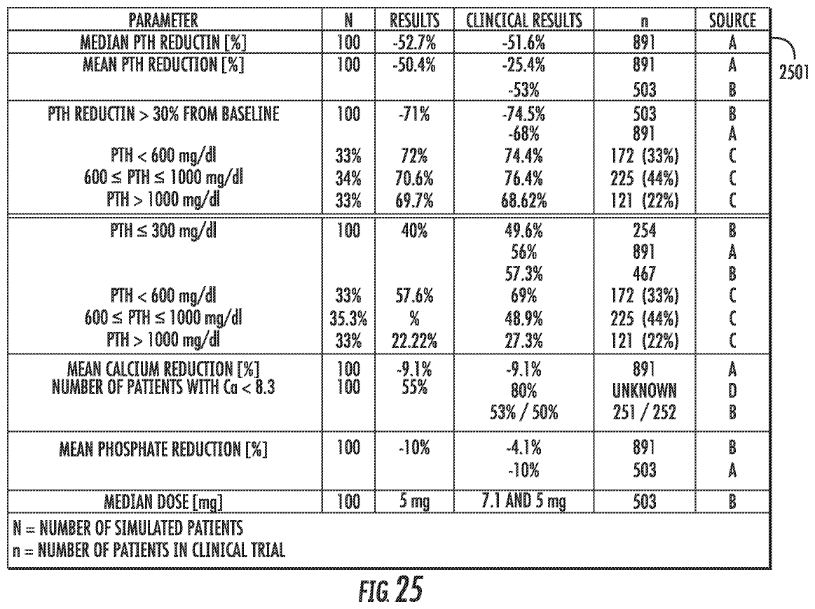

FIG. 25 depicts calcimimetic model information according to some embodiments. More specifically, FIG. 25 includes Table 2501 providing a comparison of simulation results generated via phosphate-adjusted calcimimetic models according to some embodiments and the efficacy phase clinical studies of Source A, Source B, Source C, and Source D of FIG. 13.

A phosphate baseline concentration may have an effect on the efficacy of etelcalcetide. In some embodiments, calcimimetic model simulations may be configured to analyze the influence of the baseline phosphate on the efficacy of etelcalcetide. Accordingly, some embodiments may use a patient population configured as follows:
The population of patients (for example, N=300) may be configured such that parathyroid gland, pharmacokinetics, and pharmacodynamics parameters are fixed within a single patient;
For each patient a simulation may be run for different phosphate baseline concentrations, for example, 5.5 mg/dl, 6 mg/dl, and 6.5 mg/dl;
Phosphate baseline concentrations correlate with calcium baseline concentrations as well as PTG size. Therefore, for every phosphate concentration, a calcium concentration and a PTG growth capacity were randomly chosen which correlate with the phosphate concentration. The following simulations may then be performed: (1) medium correlation between phosphate and calcium and PTG growth capacity, (2) no correlation between calcium and phosphate and a weak correlation between phosphate and PTG growth capacity, (3) weak calcium and PTG growth capacity correlation, (4) weak correlation between calcium and phosphate and medium correlation between PTG growth capacity and phosphate.

Medium correlation between phosphate and calcium and PTG growth capacity results in the predictions provided in the following Table 6:

TABLE 6

| | Mean PTH | Mean | |
|---|---|---|---|
| Phosphate | reduction | etelcalcetide | % of patients within |
| baseline | 24-27 weeks | dose | PTH target range |
| 5.5 mg/dl | −34% | 10.5 mg | 49% |
| 6 mg/dl | −37.9% | 12.5 mg | 42% |
| 6.5 mg/dl | −36% | 13 mg | 31% |

In accordance with literature data, the mean PTH reduction is not significantly different between the three phosphate baseline strata. However, the probability of achieving the PTH target range is significantly reduced in the high phosphate baseline group. A formal logistic regression (phosphate baseline concentrations as predictor for the odds of achieving the PTH target range after 24 weeks of etelcalcetide administration) indicates that 1 mg/dl increase in baseline phosphate concentration decreases the odds of achieving the target range by 41%.

The predicted numbers may change with different (meaningful) assumptions on the correlation of phosphate with baseline calcium and growth capacity. However, the overall results may be stationary: high phosphate baseline concentrations reduce the odds of achieving the PTH target range significantly.

For example, similar simulations based on medium to strong correlations between phosphate and calcium (R²=43%) and PTG growth capacity (R²=37%) would result in the prediction that 1 mg/dl increase in baseline phosphate would decrease the odds of achieving the target range by 58%. A weaker correlation between calcium and phosphate (R²=20%) as well as a weak correlation between phosphate and the PTG growth capacity (R²=25%) would predict a decrease of achieving the PTH target range by 68% for every increase in 1 mg/dl of phosphate baseline concentrations. A weaker correlation between calcium and phosphate (R²=16%) as well as a weak correlation between phosphate and the PTG growth capacity (R²=23%) would predict a decrease of achieving the PTH target range by 53% for every increase in 1 mg/dl of phosphate baseline concentrations. A weak correlation between calcium and phosphate (R²=20%) and a weak correlation between the PTG growth capacity and baseline phosphate concentrations (R²=16%) results. A highly unrealistic case, no correlation between calcium and phosphate (R²=0.02%) as well as a medium correlation between phosphate and the PTG growth capacity (R²=37%), would result in a decrease of achieving the PTH target range by 17.4% for every increase in 1 mg/dl of phosphate baseline concentrations.

Accordingly, high phosphate baseline levels imply a significant decrease in the odds of achieving PTH target range, which may not be influenced by the choice of (meaningful) parameters. Non-meaningful parameters (i.e., no correlation between PTG growth capacity and baseline phosphate) might result in a non-significant logistic regression of the odds of achieving PTH target range with phosphate as its sole predictor.

In some embodiments, calcimimetic models may simulate etelcalcetide therapy in combination with other therapeutic compounds. For example, calcimimetic models may simulate etelcalcetide therapy in combination with cinacalcet. In various embodiments, calcimimetic models may incorporate PTG models as described in the present disclosure, for instance, the same or similar to the pharmacokinetics model of cinacalcet described in Schappacher-Tilp et al., “A Multi-Compartment Model Capturing the Pharmacokinetics of the Calcimimetic Cinacalcet,” Cellular Physiology and Biochemistry 53:429-438 (2019).

In some embodiments, the total sum of weighted drug concentrations (Cₑₜₑ+1.5·10²C_{cina}^{0.9}) is used in the PTG model. To validate the weighted drug concentration, a simulated clinical trial with cinacalcet in the absence of etelcalcetide is performed and compare the results to clinical data. In the simulated clinical trial, the cinacalcet dose was adjusted according to the following titration scheme: the starting dose was 30 mg; cinacalcet can be up-titrated every four weeks in order to reach the iPTH target; the iPTH targeted range was 150 to 300 pg/ml, as reported in the respective clinical trials; cinacalcet is on hold if total calcium concentration falls below 7.5 mg/dl; and the maximal daily dose is 180 mg. The results are provided in the following Table 7:

TABLE 7

| | | Simulated | Clinical | | |
|---|---|---|---|---|---|
| Parameter | N | Results | results | n | Source |
| PTH reduction >30% | 100 | 60% | 57.7% | 343 | E |
| from baseline | | | | | |
| PTH reduction >50% | 100 | 42% | 40.2% | 343 | E |
| from baseline | | | | | |

Source E: Block et al., “Effect of Etelcalcetide vs Placebo on Serum Parathyroid Hormone in Patients Receiving Hemodialysis With Secondary Hyperpara-thyroidism Two Randomized Clinical Trials,” JAMA-Journal of the American Medical Association, 317:146-155 (2017).

The following Table 8 provides results of simulation of etelcalcetide (E) and cinacalcet (C) administration using calcimimetic models according to some embodiments:

TABLE 8

| | Administration (Monday\|Wednesday\|Friday) | | | |
|---|---|---|---|---|
| Within target | C\|C\|E | E\|E\|E | —\|—\|E | C\|C\|E(no titration) |
| PTH (150-600 pg/ml) | 19.7% | 17.7% | 12.3% | 18% |
| P (3-5.5 mg/dl) | 73.3% | 74.4% | 53.7% | 72.7% |
| Ca (8.5-10 mg/dl) | 65% | 64.7% | 69.7% | 67.3% |

For the combined etelcalcetide and cinacalcet simulation, the following therapies were simulated for each patient in the patient population (N=300):
Thrice weekly etelcalcetide starting the first dose on Fridays;
Once weekly etelcalcetide every Friday;
Once weekly etelcalcetide every Friday and cinacalcet on Mondays and Wednesday. In this scenario dose titration was allowed for etelcalcetide and cinacalcet every 4 weeks; and
Once weekly etelcalcetide every Friday and cinacalcet on Mondays and Wednesday. To simplify a possible implementation in the clinics only dose up-titration of cinacalcet was allowed. Etelcalcetide dose was down-titrated in case of a hypocalcemic condition (serum calcium below 8.5 mg/dl).

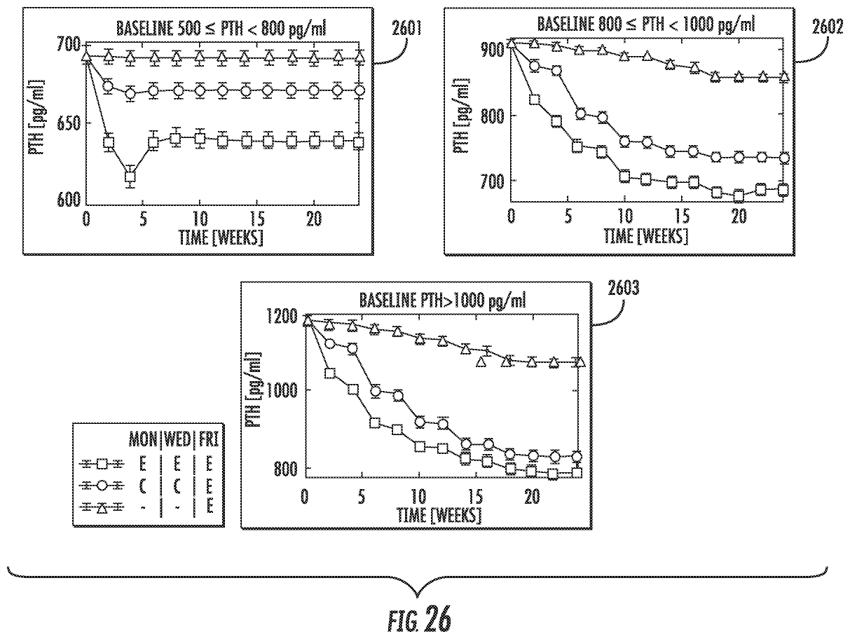

FIG. 26 depicts calcimimetic information generated via calcimimetic models according to some embodiments. More specifically, FIG. 26 depicts graphs 2601-2603 for PTH based on simulations generated via combined etelcalcetide and cinacalcet according to various embodiments. In general, FIG. 26 indicates the effect of the combination of etelcalcetide and cinacalcet in three PTH baseline concentration strata. Thrice weekly etelcalcetide is most efficient. While once weekly etelcalcetide is least efficient in controlling PTH. The difference between once weekly etelcalcetide and twice weekly cinacalcet is purely due to the administration of cinacalcet. The difference between thrice weekly and the combination therapy is small.

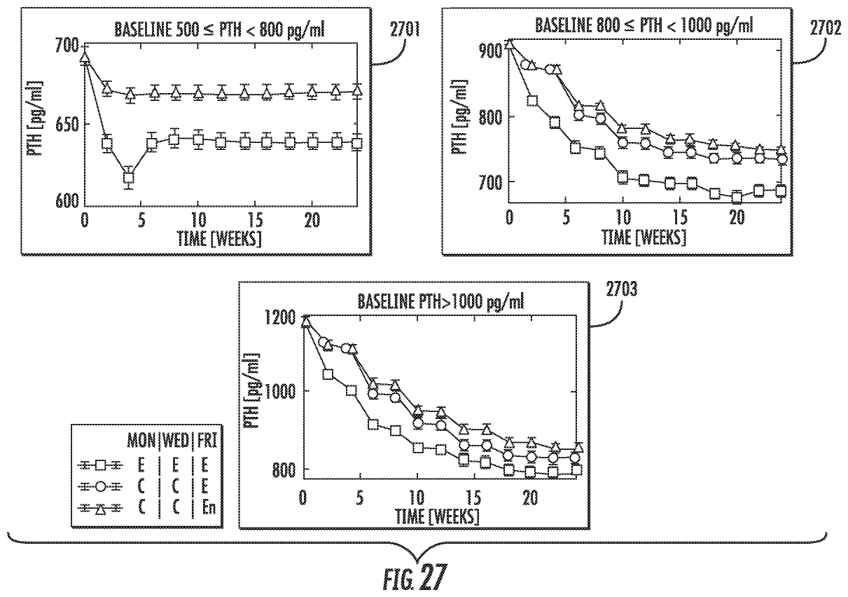

FIG. 27 depicts calcimimetic information generated via calcimimetic models according to some embodiments. More specifically, FIG. 27 depicts graphs 2701-2703 for PTH based on simulations generated via combined etelcalcetide and cinacalcet according to various embodiments. In general, the results depicted in FIG. 27 indicate the effect of the combination of etelcalcetide and cinacalcet in three PTH baseline concentration strata. Thrice weekly etelcalcetide is most efficient. Etelcalcetide and cinacalcet where both up-titrated for twice weekly cinacalcet, only cinacalcet was allowed to be up-titrated for the blue line. The difference between the mean values after 24 weeks treatment is within 10 pg/ml.

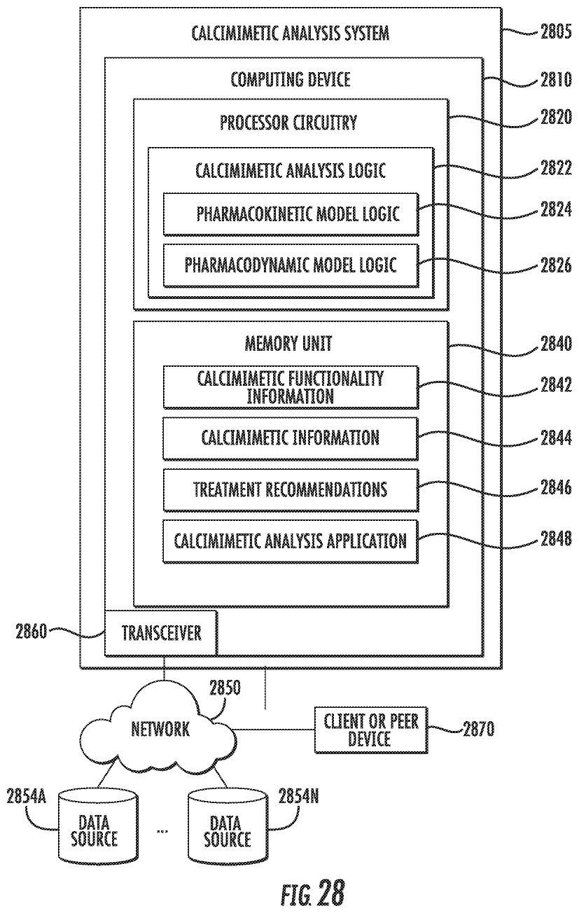

FIG. 28 illustrates an example of an operating environment 2800 that may be representative of some embodiments. As shown in FIG. 28, operating environment 2800 may include a calcimimetic analysis system 2805. In various embodiments, calcimimetic analysis system 2805 may include a computing device 2810 communicatively coupled to a network 2850 via a transceiver 2860. In some embodiments, computing device 2810 may be a server computer or other type of computing device.

Computing device 2810 may be configured to manage, among other things, operational aspects of a calcimimetic analysis process according to some embodiments. Although only one computing device 2810 is depicted in FIG. 28, embodiments are not so limited. In various embodiments, the functions, operations, configurations, data storage functions, applications, logic, and/or the like described with respect to computing device 2810 may be performed by and/or stored in one or more other computing devices (not shown), for example, coupled to computing device 2810 via network 2850 (for instance, one or more of client or peer devices 2870). A single computing device 2810 is depicted for illustrative purposes only to simplify the figure. Embodiments are not limited in this context.

Computing device 2810 may include a processor circuitry that may include and/or may access various logics for performing processes according to some embodiments. For instance, processor circuitry 2820 may include and/or may access a calcimimetic analysis logic 2822, pharmacokinetic model logic 2824, and/or pharmacodynamic model logic 2826. Processing circuitry 2820, calcimimetic analysis logic 2822, pharmacokinetic model logic 2824, and/or pharmacodynamic model logic 2826, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 3100. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, variations thereof, combinations of any of the foregoing, and/or the like.

Although calcimimetic analysis logic 2822 is depicted in FIG. 28 as being within processor circuitry 2820, embodiments are not so limited. For example, calcimimetic analysis logic 2822, pharmacokinetic model logic 2824, pharmacodynamic model logic 2826, and/or any component thereof may be located within an accelerator, a processor core, an interface, an individual processor die, implemented entirely as a software application (for instance, an calcimimetic analysis application 2840) and/or the like.

Memory unit 2840 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 2840 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 2840 may store various types of information and/or applications for a calcimimetic analysis process according to some embodiments. For example, memory unit 2840 may store calcimimetic functionality information 2842, calcimimetic information 2844, treatment recommendations 2846, and/or a calcimimetic analysis application 2848. In some embodiments, some or all of calcimimetic functionality information 2842, calcimimetic information 2844, treatment recommendations 2846, and/or a calcimimetic analysis application 2848 may be stored in one or more data stores 2854a-n accessible to computing device 2810 via network 2850. For example, one or more of data stores 2854a-n may be or may include a HIS, an EMR system, a dialysis information system (DIS), a picture archiving and communication system (PACS), a Centers for Medicare and Medicaid Services (CMS) database, U.S. Renal Data System (USRDS), a proprietary database, and/or the like.

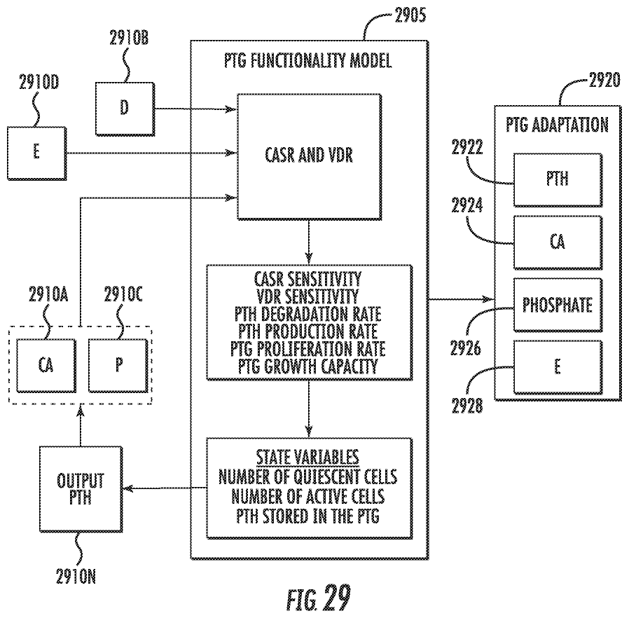

In some embodiments, calcimimetic analysis logic 2822, for example, via pharmacokinetic model logic 2824, pharmacodynamic model logic 2826, and/or calcimimetic analysis application 2848, may operate to simulate the pharmacokinetic processes and/or pharmacodynamic processes of etelcalcetide administration according to various embodiments described in the present disclosure. FIG. 29 depicts the functionality of illustrative calcimimetic models according to some embodiments. As shown in FIG. 29, a calcimimetic model 2905 operative to receive a plurality of inputs or parameters 2910a-n associated with the functionality of various aspects of the calcimimetic model and to generate an output 2920. In various embodiments, inputs 2910a-n may include a calcium concentration 2910a, a vitamin D (1.25D) concentration 2910b, a phosphate concentration 2910c, an etelcalcetide concentration 210d (for instance, via a calcimimetic model according to some embodiments). In some embodiments, Ca2+, 1.25D, and phosphate profiles used in PTH predictions in calcimimetic model 2905 may be simulated profiles. In various embodiments, the in-silico data that is used as input 2910a-n to PTG functionality model 2905 may be within physiological meaningful ranges.

In exemplary embodiments, output 2920 may be a PTG adaptation associated with the major adaptation mechanisms governing the production and secretion of PTH for patients, for example, healthy patients and/or patients with health abnormalities that affect PTH functionality, such as patients with CKD on HD. In various embodiments, output 2920 may be a PTH concentration 2922, a calcium concentration 224, a phosphate concentration 2926, and/or an etelcalcetide concentration 2928.

In reference to FIG. 28, calcimimetic functionality information 2842 may include information associated with calcimimetic analysis logic 2822, such as input information, parameters, models, model structures, outputs, thresholds, limits, constants, algorithms, equations, and/or the like. Embodiments are not limited in this context.

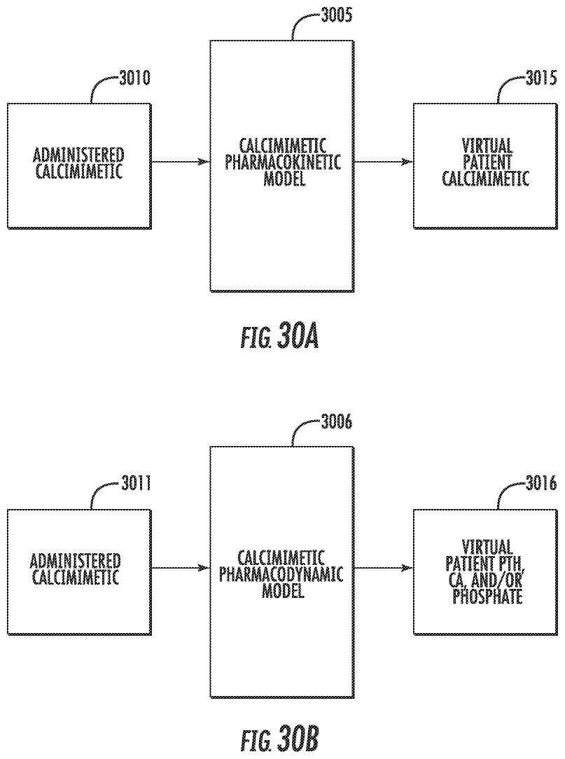

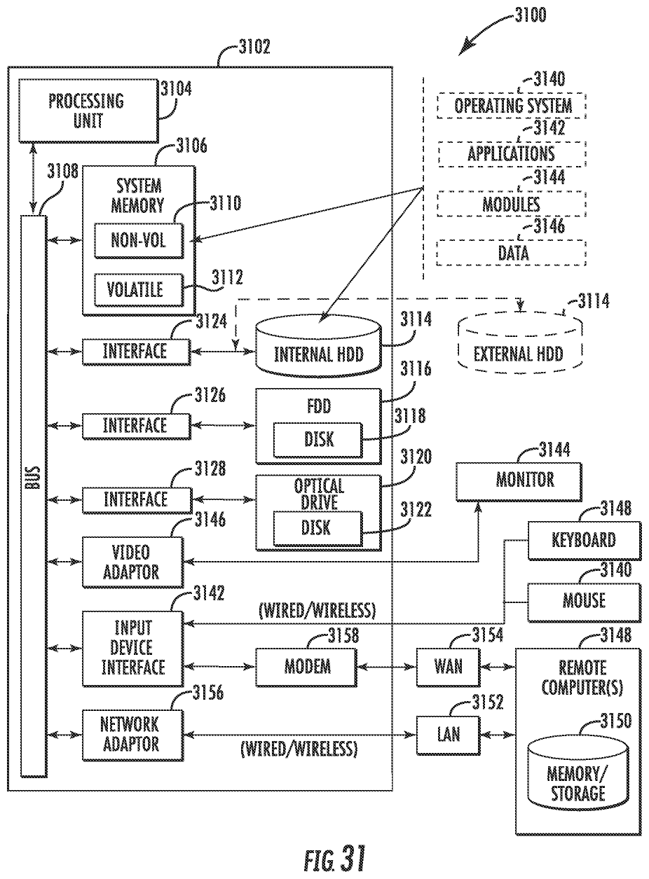

In some embodiments, calcimimetic analysis logic 2822, for example, via pharmacokinetic model logic 2824, pharmacodynamic model logic 2826, and/or PTG analysis application 2848, may operate to simulate etelcalcetide administration via a calcimimetic model. FIGS. 30A and 30B depict illustrative calcimimetic models according to some embodiments. Referring to FIG. 30A, therein is depicted calcimimetic pharmacokinetic model 3005 configured to receive an administered calcimimetic concentration 3010 as input and to generate a virtual patient calcimimetic concentration 3015 as an output. In various embodiments, the calcimimetic may be or may include etelcalcetide. In some embodiments, the calcimimetic may be or may include cinacalcet.

Referring to FIG. 30B, therein is depicted calcimimetic pharmacodynamics model 3006 according to some embodiments configured to receive an administered calcimimetic concentration 3011 as input and to generate a virtual patient PTH, Ca, and/or phosphate 3016 as an output. In various embodiments, the calcimimetic may be or may include etelcalcetide. In some embodiments, the calcimimetic may be or may include cinacalcet.

FIG. 31 illustrates an embodiment of an exemplary computing architecture 3100 suitable for implementing various embodiments as previously described. In various embodiments, the computing architecture 3100 may comprise or be implemented as part of an electronic device. In some embodiments, the computing architecture 3100 may be representative, for example, of computing device 110. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 3100. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 3100 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 3100.

As shown in FIG. 31, the computing architecture 3100 comprises a processing unit 3104, a system memory 3106 and a system bus 3108. The processing unit 3104 may be a commercially available processor and may include dual microprocessors, multi-core processors, and other multi-processor architectures.

The system bus 3108 provides an interface for system components including, but not limited to, the system memory 3106 to the processing unit 3104. The system bus 3108 can be any of several types of bus structure that may further interconnect to a memory bus (with or without a memory controller), a peripheral bus, and a local bus using any of a variety of commercially available bus architectures. Interface adapters may connect to the system bus 3108 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 3106 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 31, the system memory 3106 can include non-volatile memory 3110 and/or volatile memory 3112. A basic input/output system (BIOS) can be stored in the non-volatile memory 3110. The computer 3102 may include various types of computer-readable storage media in the form of one or more lower speed memory units, including an internal (or external) hard disk drive (HDD) 3114, a magnetic floppy disk drive (FDD) 3116 to read from or write to a removable magnetic disk 3111, and an optical disk drive 3120 to read from or write to a removable optical disk 3122 (e.g., a CD-ROM or DVD). The HDD 3114, FDD 3116 and optical disk drive 3120 can be connected to the system bus 3108 by a HDD interface 3124, an FDD interface 3126 and an optical drive interface 3128, respectively. The HDD interface 3124 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 3110, 3112, including an operating system 3130, one or more application programs 3132, other program modules 3134, and program data 3136. In one embodiment, the one or more application programs 3132, other program modules 3134, and program data 3136 can include, for example, the various applications and/or components of computing device 110.

A user can enter commands and information into the computer 3102 through one or more wired/wireless input devices, for example, a keyboard 3138 and a pointing device, such as a mouse 3140. These and other input devices are often connected to the processing unit 3104 through an input device interface 3142 that is coupled to the system bus 3108, but can be connected by other interfaces.

A monitor 3144 or other type of display device is also connected to the system bus 3108 via an interface, such as a video adaptor 3146. The monitor 3144 may be internal or external to the computer 3102. In addition to the monitor 3144, a computer typically includes other peripheral output devices, such as speakers, printers, and so forth.

The computer 3102 may operate in a networked environment using logical connections via wired and/or wireless communications to one or more remote computers, such as a remote computer 3148. The remote computer 3148 can be a workstation, a server computer, a router, a personal computer, portable computer, microprocessor-based entertainment appliance, a peer device or other common network node, and typically includes many or all of the elements described relative to the computer 3102, although, for purposes of brevity, only a memory/storage device 3150 is illustrated. The logical connections depicted include wired/wireless connectivity to a local area network (LAN) 3152 and/or larger networks, for example, a wide area network (WAN) 3154. Such LAN and WAN networking environments are commonplace in offices and companies, and facilitate enterprise-wide computer networks, such as intranets, all of which may connect to a global communications network, for example, the Internet.

The computer 3102 is operable to communicate with wired and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A computer-implemented method of calcimimetic activity analysis of the parathyroid gland (PTG), the method comprising, via a processor of a computing device:
accessing a calcimimetic model configured to simulate a functionality of a PTG of at least one patient, the calcimimetic model comprising at least one of a pharmacokinetic model or a pharmacodynamic model;
providing a calcimimetic administration of a calcimimetic to the at least one patient via the calcimimetic model according to an administration process; and
determining calcimimetic information based on the calcimimetic administration via the calcimimetic model for the at least one patient, the calcimimetic information configured to indicate an efficacy of the calcimimetic administration.

2. The computer-implemented method of claim 1, the calcimimetic comprising etelcalcetide.

3. The computer-implemented method of claim 1, the calcimimetic administration comprising at least one dose titration process for the calcimimetic.

4. The computer-implemented method of claim 1, the at least one dose titration process comprising one or more of adjusting a dose of the calcimimetic on a constant time span, holding calcimimetic administration responsive to a calcium concentration being below a hold threshold, reducing a dose of the calcimimetic responsive to a calcium concentration being below a reduce threshold, or raising a calcimimetic dose responsive to a PTH concentration being within a threshold range.

5. The computer-implemented method of claim 1, the pharmacokinetic model configured to simulate pharmacokinetic functionality of the calcimimetic for the at least one patient, the calcimimetic information for the pharmacokinetic model comprising a calcimimetic concentration.

6. The computer-implemented method of claim 5, the calcimimetic comprising etelcalcetide and the calcimimetic information for the pharmacokinetic model comprising at least one of intact etelcalcetide, etelcalcetide biotransforms, or peripheral compartment etelcalcetide.

7. The computer-implemented method of claim 6, the calcimimetic information determined according to at least one of the following:
$$\frac{d {C}_{i}}{d t} = - ( \frac{C {L}_{u}}{{V}_{i}} + \frac{{C}_{e l}}{{V}_{i}} + {k}_{c} + {χ}_{H D} \cdot \frac{Q}{{V}_{i}} \cdot E ) \cdot {C}_{p} + {k}_{d c} \cdot {C}_{B i o} \\ \frac{d {C}_{B i o}}{d t} = - ( \frac{C {L}_{u}}{{V}_{b i o}} \cdot Φ + \frac{{C}_{f}}{{V}_{b i o}} + {k}_{p t} + {k}_{d c} + {χ}_{H D} \cdot \frac{Q}{{V}_{b i o}} \cdot E \cdot Φ ) \cdot {C}_{B i o} + {k}_{c} \cdot {C}_{i} + {k}_{t p} \cdot {C}_{p e r} \\ \frac{d {C}_{p e r}}{d t} = - {k}_{t p} \cdot {C}_{per} + {k}_{p t} \cdot {C}_{B i o} ,$$
where C denotes a calcimimetic, i denotes intact calcimimetic, bio denotes calcimimetic biotransforms, and per denotes a peripheral compartment,
where Q is the plasma flow during dialysis,
where Φ denotes a fraction of filterable biotransforms,
where E is a dialysis extraction ratio for the calcimimetic,
where Vᵢ is a volume of distribution in a central compartments for intact calcimimetic,
where V_{bio} is a volume of distribution in the central compartments for calcimimetic biotransforms,
where χ_{HD}=1 if there is a dialysis session ongoing and χ_{HD}=0 if there is not a dialysis session ongoing,
where k_{c} is a conjugation rate constant,
where k_{dc} is a deconjugation rate constant,
where kₚₜ is a transfer from plasma to peripheral compartment rate constant,
where kₜₚ is a transfer from peripheral compartment to central compartment rate constant,
where C_{f} is an elimination rate of intact calcimimetic in feces,
where CLᵤ is a urinal clearance, and
where Cₑₗ is an elimination rate of intact calcimimetic in plasma.

8. The computer-implemented method of claim 1, the pharmacodynamic model configured to simulate pharmacodynamic functionality of the calcimimetic for the at least one patient, the calcimimetic information for the pharmacokinetic model comprising at least one of a parathyroid (PTH) concentration, a calcium concentration, or a phosphate concentration.

9. The computer-implemented method of claim 1, comprising determining at least one treatment recommendation based on the calcimimetic information.

10. The computer-implemented method of claim 1, comprising determining at least one clinical trial based on the calcimimetic information.

11. An apparatus, comprising:
at least one processor; and
a memory coupled to the at least one processor, the memory comprising instructions that, when executed by the at least one processor, cause the at least one processor to:
access a calcimimetic model configured to simulate a functionality of a PTG of at least one patient, the calcimimetic model comprising at least one of a pharmacokinetic model or a pharmacodynamic model,
provide a calcimimetic administration of a calcimimetic to the at least one patient via the calcimimetic model according to an administration process, and
determine calcimimetic information based on the calcimimetic administration via the calcimimetic model for the at least one patient, the calcimimetic information configured to indicate an efficacy of the calcimimetic administration.

12. The apparatus of claim 11, the calcimimetic comprising etelcalcetide.

13. The apparatus of claim 11, the calcimimetic administration comprising at least one dose titration process for the calcimimetic.

14. The apparatus of claim 11, the at least one dose titration process may include one or more of adjusting a dose of the calcimimetic on a constant time span, holding calcimimetic administration responsive to a calcium concentration being below a hold threshold, reducing a dose of the calcimimetic responsive to a calcium concentration being below a reduce threshold, or raising a calcimimetic dose responsive to a PTH concentration being within a threshold range.

15. The apparatus of claim 11, the pharmacokinetic model configured to simulate pharmacokinetic functionality of the calcimimetic for the at least one patient, the calcimimetic information for the pharmacokinetic model comprising a calcimimetic concentration.

16. The apparatus of claim 15, the calcimimetic comprising etelcalcetide and the calcimimetic information for the pharmacokinetic model comprising at least one of intact etelcalcetide, etelcalcetide biotransforms, or peripheral compartment etelcalcetide.

17. The apparatus of claim 16, the calcimimetic information determined according to at least one of the following:
$$\frac{d {C}_{i}}{d t} = - ( \frac{C {L}_{u}}{{V}_{i}} + \frac{{C}_{e l}}{{V}_{i}} + {k}_{c} + {χ}_{H D} \cdot \frac{Q}{{V}_{i}} \cdot E ) \cdot {C}_{p} + {k}_{d c} \cdot {C}_{B i o} \\ \frac{d {C}_{B i o}}{d t} = - ( \frac{C {L}_{u}}{{V}_{b i o}} \cdot Φ + \frac{{C}_{f}}{{V}_{b i o}} + {k}_{p t} + {k}_{d c} + {χ}_{H D} \cdot \frac{Q}{{V}_{b i o}} \cdot E \cdot Φ ) \cdot {C}_{B i o} + {k}_{c} \cdot {C}_{i} + {k}_{t p} \cdot {C}_{p e r} \\ \frac{d {C}_{p e r}}{d t} = - {k}_{t p} \cdot {C}_{per} + {k}_{p t} \cdot {C}_{B i o} ,$$
where C denotes a calcimimetic, i denotes intact calcimimetic, bio denotes calcimimetic biotransforms, and per denotes a peripheral compartment,
where Q is the plasma flow during dialysis,
where Φ denotes a fraction of filterable biotransforms,
where E is a dialysis extraction ratio for the calcimimetic,
where Vᵢ is a volume of distribution in a central compartments for intact calcimimetic,
where V_{bio} is a volume of distribution in the central compartments for calcimimetic biotransforms,
where χ_{HD}=1 if there is a dialysis session ongoing and χ_{HD}=0 if there is not a dialysis session ongoing,
where k_{c} is a conjugation rate constant,
where k_{dc} is a deconjugation rate constant,
where kₚₜ is a transfer from plasma to peripheral compartment rate constant,
where kₜₚ is a transfer from peripheral compartment to central compartment rate constant,
where C_{f} is an elimination rate of intact calcimimetic in feces,
where CLᵤ is a urinal clearance, and
where Cₑₗ is an elimination rate of intact calcimimetic in plasma.

18. The apparatus of claim 11, the pharmacodynamic model configured to simulate pharmacodynamic functionality of the calcimimetic for the at least one patient, the calcimimetic information for the pharmacokinetic model comprising at least one of a parathyroid (PTH) concentration, a calcium concentration, or a phosphate concentration.

19. The apparatus of claim 11, the instructions, when executed by the at least one processor, to cause the at least one processor to determine at least one treatment recommendation based on the calcimimetic information.

20. The apparatus of claim 11, the instructions, when executed by the at least one processor, to cause the at least one processor to determine at least one clinical trial based on the calcimimetic information.
